(19) United States
(12) Patent Application Publication
(10) Pub. No.: US 2015/0294481 A1
(43) Pub. Date: Oct. 15, 2015

(54) MOTION INFORMATION PROCESSING APPARATUS AND METHOD

(71) Applicants: Kabushiki Kaisha Toshiba, Minato-ku (JP); Toshiba Medical Systems Corporation, Otawara-shi (JP)

(72) Inventor: Kousuke Sakaue, Nasushiobara (JP)

(73) Assignees: Kabushiki Kaisha Toshiba, Minato-ku (JP); Toshiba Medical Systems Corporation, Otawara-shi (JP)

(21) Appl. No.: 14/751,199

(22) Filed: Jun. 26, 2015

Related U.S. Application Data
(63) Continuation of application No. PCT/JP2013/085251, filed on Dec. 27, 2013.

(30) Foreign Application Priority Data
Dec. 28, 2012 (JP) 2012-288668
Jan. 18, 2013 (JP) 2013-007850

(51) Int. Cl. G06T 7/20; G06K 9/00; A61B 5/11; G06T 7/00

(52) U.S. Cl. CPC G06T 7/20; G06T 7/0028; G06T 7/004; G06T 7/0051; G06K 9/00348; A61B 5/1127; A61B 5/112; A61B 5/1116; A61B 5/1124; A61B 5/1121; G06T 2207/10016; G06T 2207/10024; G06T 2207/10028; G06T 2207/30196; G06T 2207/30204

(57) ABSTRACT

A motion information processing apparatus according to embodiments includes an acquiring unit and an output unit. The acquiring unit acquires motion information indicating a motion of a person. The output unit outputs support information used to support a motion relating to rehabilitation for the person whose motion information is acquired by the acquiring unit.

### CROSS-REFERENCE TO RELATED APPLICATIONS

This application is a continuation of PCT international application Ser. No. PCT/JP2013/085251 filed on Dec. 27, 2013 which designates the United States, incorporated herein by reference, and which claims the benefit of priority from Japanese Patent Application No. 2012-288668, filed on Dec. 28, 2012 and Japanese Patent Application No. 2013-007850, filed on Jan. 18, 2013, the entire contents of which are incorporated herein by reference.

### FIELD

Embodiments described herein relate generally to a motion information processing apparatus and a method.

### BACKGROUND

Conventionally, in rehabilitation, a number of experts provide cooperative support to enable persons who have mental and physical disabilities caused by various reasons, such as diseases, injuries, and aging, and congenital disabilities to live better lives. In rehabilitation, for example, cooperative support is provided by a number of experts, such as rehabilitation specialists, rehabilitation nurses, physical therapists, occupational therapists, speech-language-hearing therapists, clinical psychologists, prosthetists, and social workers.

In recent years, there have been developed motion capture technologies for digitally recording a motion of a person or an object. Examples of systems of the motion capture technologies include an optical, a mechanical, a magnetic, and a camera system. Widely known is the camera system for digitally recording a motion of a person by attaching markers to the person, detecting the markers with a tracker, such as a camera, and processing the detected markers, for example. Examples of systems using no marker or no tracker include a system for digitally recording a motion of a person by using an infrared sensor, measuring a distance from the sensor to the person, and detecting the size of the person and various types of motions of the skeletal structure. Examples of the sensors provided with such a system include Kinect (registered trademark).

### BRIEF DESCRIPTION OF THE DRAWINGS

FIG. 1 is a block diagram of an exemplary configuration of a motion information processing apparatus according to a first embodiment;

FIG. 2A is a view for explaining processing of a motion information generating unit according to the first embodiment;

FIG. 2B is a view for explaining processing of the motion information generating unit according to the first embodiment;

FIG. 2C is a view for explaining processing of the motion information generating unit according to the first embodiment;

FIG. 3 is a diagram of an example of skeletal information generated by the motion information generating unit according to the first embodiment;

FIG. 4 is a block diagram of a detailed exemplary configuration of the motion information processing apparatus according to the first embodiment;

FIG. 5A to FIG. 5E are diagrams of an example of object person information stored in an object person information storage unit according to the first embodiment;

FIG. 6 is a diagram of an example of rule information stored in a rule information storage unit according to the first embodiment;

FIG. 7 is a view for explaining an example of determination processing performed by a determining unit according to the first embodiment;

FIG. 8 is a flowchart of a procedure of processing performed by the motion information processing apparatus according to the first embodiment;

FIG. 9 is a view for explaining an example of processing performed by a determining unit according to a second embodiment;

FIG. 10 is a view for explaining an example of determination processing performed by a determining unit according to a third embodiment;

FIG. 11 is a schematic of an example of a distance image captured by a distance image acquiring unit;

FIG. 12 is a block diagram of a detailed exemplary configuration of a motion information processing apparatus according to a fifth embodiment;

FIG. 13A is a diagram of an example of information stored in an object person motion characteristic storage unit;

FIG. 13B is a diagram of an example of information stored in an assistant motion characteristic storage unit;

FIG. 13C is a diagram of an example of information stored in an object person image characteristic storage unit;

FIG. 13D is a diagram of an example of information stored in an assistant image characteristic storage unit;

FIG. 14A is a diagram of an example of information stored in a first mode determination storage unit;

FIG. 14B is a diagram of an example of information stored in a second mode determination storage unit;

FIG. 15 is a diagram of an example of information stored in a recommended assistance state storage unit;

FIG. 16A is a view for explaining determination processing performed by a person determining unit based on positions of persons;

FIG. 16B is a view for explaining determination processing performed by the person determining unit using an identification marker;

FIG. 17A is a view for explaining processing of a mode determining unit;

FIG. 17B is a view for explaining processing of the mode determining unit;

FIG. 17C is a view for explaining processing of the mode determining unit;

FIG. 17D is a view for explaining processing of the mode determining unit;

FIG. 17E is a view for explaining processing of the mode determining unit;

FIG. 18A is a view for explaining processing of a detecting unit;

FIG. 18B is a view for explaining processing of the detecting unit;

FIG. 18C is a view for explaining processing of the detecting unit;

FIG. 19A is a view for explaining processing of an output determining unit;

FIG. 19B is a view for explaining processing of the output determining unit;

FIG. 20 is a flowchart for explaining an example of a processing procedure of the motion information processing apparatus according to the fifth embodiment;

FIG. 21 is a flowchart for explaining an example of a processing procedure of person determination processing according to the fifth embodiment;

FIG. 22 is a view for explaining advantageous effects of the motion information processing apparatus according to the fifth embodiment;

FIG. 23 is a view for explaining a case where an assistant helps an object person with a standing-up motion using an assistance belt;

FIG. 24 is a diagram of an example of information stored in a recommended assistance state storage unit according to a sixth embodiment;

FIG. 25 is a diagram of an example of an entire configuration of a motion information processing apparatus according to a seventh embodiment;

FIG. 26 is a block diagram of an exemplary configuration of the motion information processing apparatus according to the seventh embodiment;

FIG. 27 is a view for explaining processing of an output control unit according to the seventh embodiment;

FIG. 28 is a view for explaining processing of an output control unit according to an eighth embodiment; and

FIG. 29 is a view for explaining an example of a case where the embodiments are applied to a service providing apparatus.

### DETAILED DESCRIPTION

According to an embodiment, a motion information processing apparatus includes processing circuitry. The processing circuitry configured to acquire motion information indicating a motion of a person. The processing circuitry configured to output support information used to support a motion relating to rehabilitation for the person whose motion information is acquired.

Exemplary embodiments of a motion information processing apparatus and a method are described below with reference to the accompanying drawings. Motion information processing apparatuses described below may be used alone or in a manner incorporated in a system, such as a medical chart system and a rehabilitation section system.

### First Embodiment

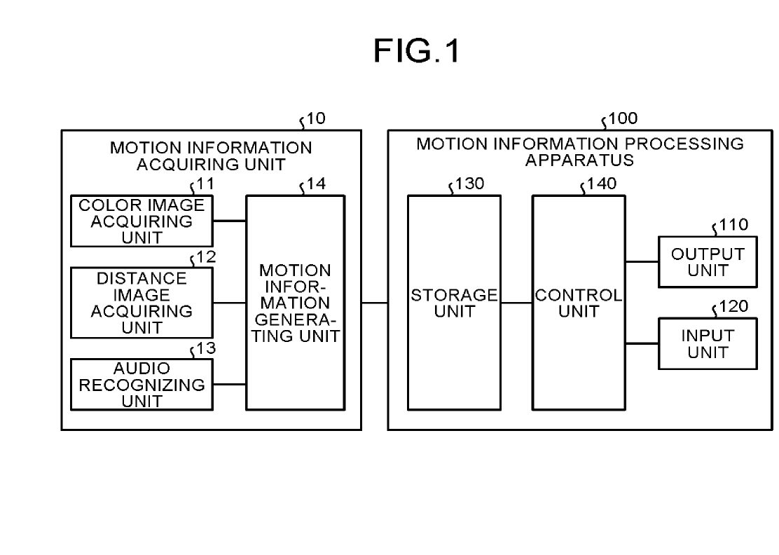

FIG. 1 is a block diagram of an exemplary configuration of a motion information processing apparatus 100 according to a first embodiment. The motion information processing apparatus 100 according to the first embodiment is an apparatus that supports rehabilitation performed at medical institutions, home, and offices, for example. “Rehabilitation” means a technology and a method for enhancing potential of patients receiving long-term treatment for disabilities, chronic diseases, geriatric diseases, and the like to restore and improve vital functions and social functions of the patients. Such a technology and a method include functional training to restore and improve vital functions and social functions, for example. Examples of the functional training include gait training and range of joint motion exercises. A person serving as a target of rehabilitation is referred to as an “object person”. Examples of the object person include sick persons, injured persons, elderly persons, and disabled persons. A person who assists the object person in rehabilitation is referred to as an “assistant”. Examples of the assistant include medical professionals who work for medical institutions, such as doctors, physical therapists, and nurses, and care workers, families, and friends who care for the object person at home. Rehabilitation may be simply referred to as “rehab”.

As illustrated in FIG. 1, the motion information processing apparatus 100 is connected to a motion information acquiring unit 10 in the first embodiment.

The motion information acquiring unit 10 detects a motion of a person, an object, or the like in a space where rehabilitation is performed, thereby acquiring motion information indicating the motion of the person, the object, or the like. The motion information will be described in detail in an explanation of processing of a motion information generating unit 14, which will be described later. The motion information acquiring unit 10 is Kinect (registered trademark), for example.

As illustrated in FIG. 1, the motion information acquiring unit 10 includes a color image acquiring unit 11, a distance image acquiring unit 12, an audio recognizing unit 13, and the motion information generating unit 14. The configuration of the motion information acquiring unit 10 illustrated in FIG. 1 is given by way of example, and the embodiment is not limited thereto.

The color image acquiring unit 11 captures a photographic subject, such as a person and an object, in a space where rehabilitation is performed, thereby acquiring color image information. The color image acquiring unit 11, for example, detects light reflected by the surface of the photographic subject with a light receiving element and converts visible light into an electrical signal. The color image acquiring unit 11 then converts the electrical signal into digital data, thereby generating color image information of one frame corresponding to a capturing range. The color image information of one frame includes capturing time information and information in which each pixel contained in the frame is associated with an RGB (red, green, and blue) value, for example. The color image acquiring unit 11 generates color image information of a plurality of consecutive frames from visible light sequentially detected, thereby capturing the capturing range as video. The color image information generated by the color image acquiring unit 11 may be output as a color image in which the RGB values of respective pixels are arranged on a bit map. The color image acquiring unit 11 includes a complementary metal oxide semiconductor (CMOS) and a charge coupled device (CCD) as the light receiving element, for example.

The distance image acquiring unit 12 captures a photographic subject, such as a person and an object, in a space where rehabilitation is performed, thereby acquiring distance image information. The distance image acquiring unit 12, for example, irradiates the surroundings with infrared rays and detects reflected waves, which are irradiation waves reflected by the surface of the photographic subject, with a light receiving element. The distance image acquiring unit 12 then derives a distance between the photographic subject and the distance image acquiring unit 12 based on the phase difference between the irradiation waves and the reflected waves and a time from the irradiation to the detection. The distance image acquiring unit 12 thus generates distance image information of one frame corresponding to the capturing range. The distance image information of one frame includes capturing time information and information in which each pixel contained in the capturing range is associated with a distance between the photographic subject corresponding to the pixel and the distance image acquiring unit 12, for example. The distance image acquiring unit 12 generates distance image information of a plurality of consecutive frames from reflected waves sequentially detected, thereby capturing the capturing range as video. The distance image information generated by the distance image acquiring unit 12 may be output as a distance image in which the gray scales of colors corresponding to the distances of the respective pixels are arranged on a bit map. The distance image acquiring unit 12 includes a CMOS and a CCD as the light receiving element, for example. The light receiving element may be shared by the color image acquiring unit 11. The unit of distance calculated by the distance image acquiring unit 12 is the meter (m), for example.

The audio recognizing unit 13 collects audio of the surroundings, identifies the direction of a sound source, and recognizes the audio. The audio recognizing unit 13 includes a microphone array provided with a plurality of microphones and performs beam forming. Beam forming is a technology for selectively collecting audio travelling in a specific direction. The audio recognizing unit 13, for example, performs beam forming with the microphone array, thereby identifying the direction of a sound source. The audio recognizing unit 13 uses a known audio recognition technology, thereby recognizing a word from the collected audio. In other words, the audio recognizing unit 13 generates information in which a word recognized by the audio recognition technology, the direction in which the word is output, and time at which the word is recognized are associated with one another as an audio recognition result, for example.

The motion information generating unit 14 generates motion information indicating a motion of a person, an object, or the like. The motion information is generated by considering a motion (gesture) of a person as a plurality of successive postures (poses), for example. Specifically, the motion information generating unit 14 performs pattern matching using a human body pattern. The motion information generating unit 14 acquires coordinates of respective joints forming a skeletal structure of a human body from the distance image information generated by the distance image acquiring unit 12. The coordinates of respective joints obtained from the distance image information are values represented by a coordinate system of a distance image (hereinafter, referred to as a “distance image coordinate system”). The motion information generating unit 14 then converts the coordinates of respective joints in the distance image coordinate system into values represented by a coordinate system of a three-dimensional space in which rehabilitation is performed (hereinafter, referred to as a “world coordinate system”). The coordinates of respective joints represented by the world coordinate system correspond to skeletal information of one frame. Skeletal information of a plurality of frames corresponds to motion information. The processing of the motion information generating unit 14 according to the first embodiment will be specifically described.

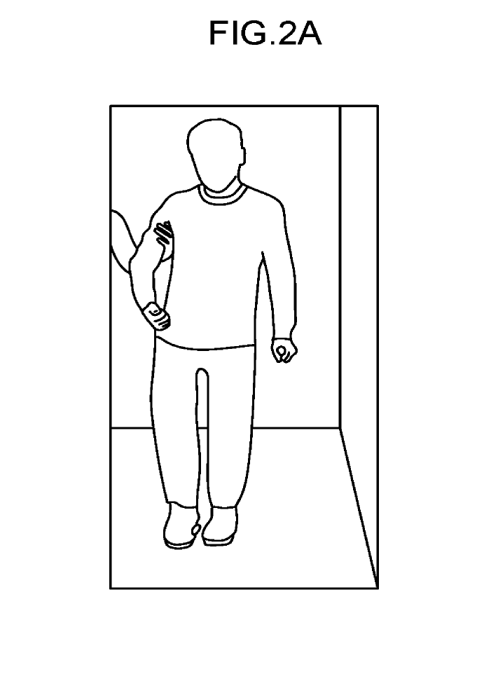

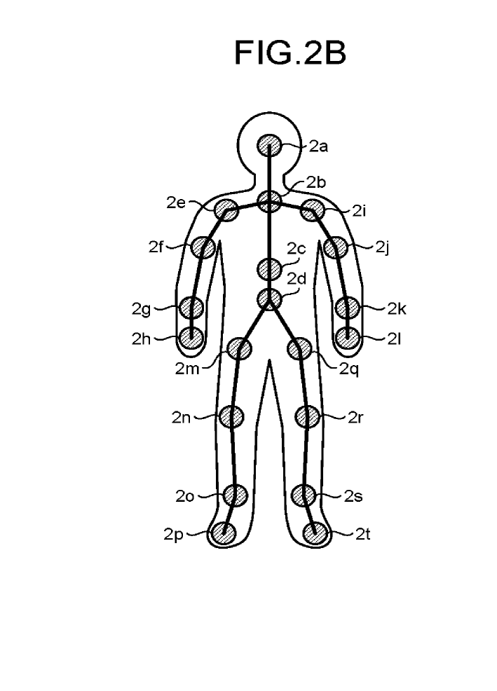

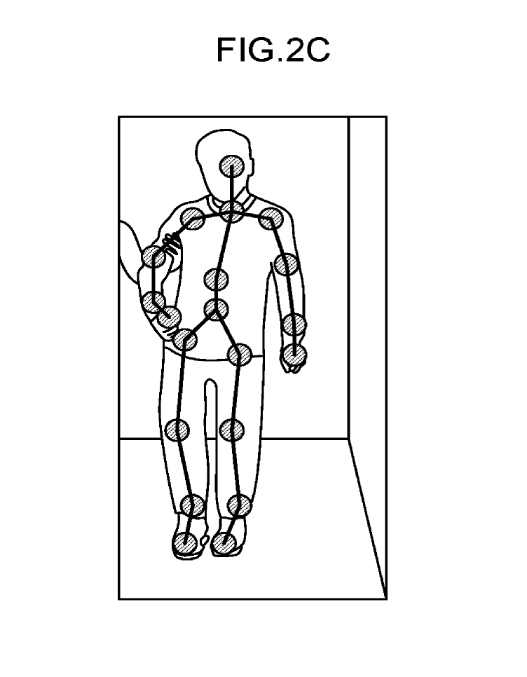

FIG. 2A to FIG. 2C are views for explaining the processing of the motion information generating unit 14 according to the first embodiment. FIG. 2A illustrates an example of a distance image generated by the distance image acquiring unit 12. While FIG. 2A illustrates an image depicted with lines for convenience of explanation, an actual distance image is an image represented by the gray scales of colors corresponding to distances, for example. In the distance image, each pixel has a three-dimensional value in which a “pixel position X” in the horizontal direction of the distance image, a “pixel position Y” in the vertical direction of the distance image, and a “distance Z” between the photographic subject corresponding to the pixel and the distance image acquiring unit 12 are associated with one another. A coordinate value in the distance image coordinate system is hereinafter represented by the three-dimensional value (X, Y, Z).

In the first embodiment, the motion information generating unit 14 stores therein in advance a human body pattern corresponding to various postures by learning, for example. Every time the distance image acquiring unit 12 generates distance image information, the motion information generating unit 14 acquires the generated distance image information of each frame. The motion information generating unit 14 then performs pattern matching of the human body pattern with the acquired distance image information of each frame.

The human body pattern will now be described. FIG. 2B illustrates an example of the human body pattern. In the first embodiment, the human body pattern is a pattern used for pattern matching with the distance image information. The human body pattern is represented by the distance image coordinate system and has information on the surface of a human body (hereinafter, referred to as a “human body surface”) similarly to the person depicted on the distance image. The human body surface corresponds to the skins of the person and the surfaces of clothes, for example. As illustrated in FIG. 2B, the human body pattern has information on joints forming the skeletal structure of the human body. In other words, a relative positional relation between the human body surface and each joint in the human body pattern is known.

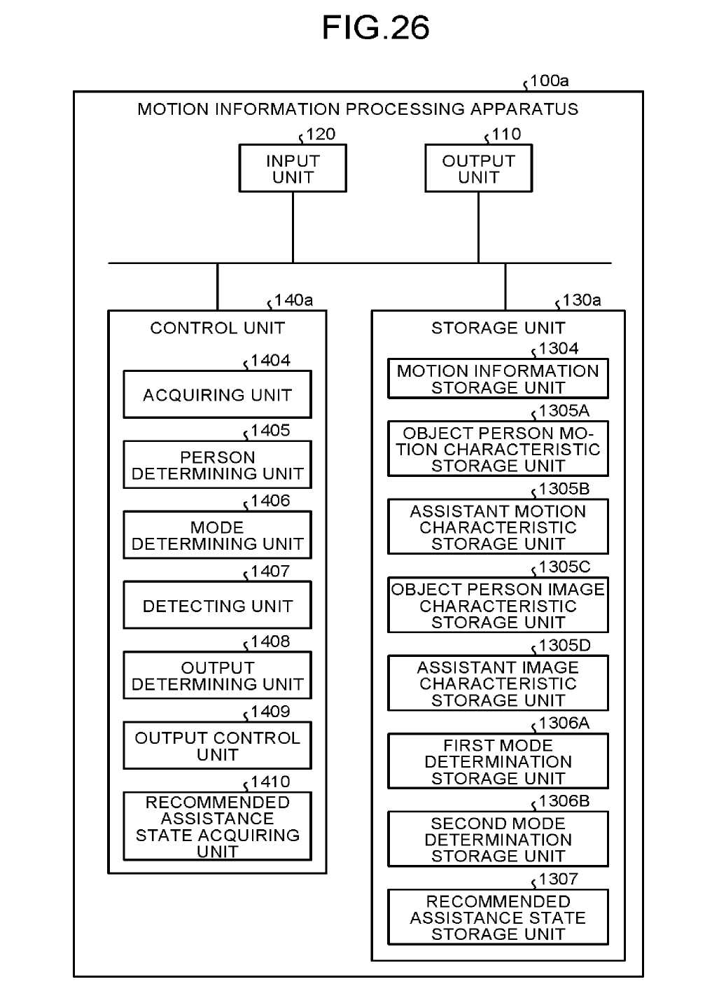

In the example of FIG. 26, the human body pattern has information on 20 joints from a joint 2a to a joint 2t. The joint 2a corresponds to the head, the joint 2b corresponds to the intermediate portion between the shoulders, the joint 2c corresponds to the waist, and the joint 2d corresponds to the center portion of the buttocks. The joint 2e corresponds to the right shoulder, the joint 2f corresponds to the right elbow, the joint 2g corresponds to the right wrist, and the joint 2h corresponds to the right hand. The joint 2i corresponds to the left shoulder, the joint 2j corresponds to the left elbow, the joint 2k corresponds to the left wrist, and the joint 21 corresponds to the left hand. The joint 2m corresponds to the right buttock, the joint 2n corresponds to the right knee, the joint 2o corresponds to the right ankle, and the joint 2p corresponds to the tarsus of the right foot. The joint 2q corresponds to the left buttock, the joint 2r corresponds to the left knee, the joint 2s corresponds to the left ankle, and the joint 2t corresponds to the tarsus of the left foot.

While the explanation has been made of the case where the body pattern has the information on 20 joints in FIG. 2B, the embodiment is not limited thereto. The positions and the number of joints may be optionally set by an operator. To grasp a change in a motion of the four limbs alone, for example, the information on the joint 2b and the joint 2c need not be acquired out of the joint 2a to the joint 2d. To grasp a change in a motion of the right hand in detail, joints of the fingers of the right hand may be further set besides the joint 2h. The joint 2a, the joint 2h, the joint 21, the joint 2p, and the joint 2t in FIG. 2B correspond to distal ends of bones and are different from what is called a joint. Because the joints 2a, 2h, 21, 2p, and 2t are important points indicating the positions and the directions of the bones, the joints 2a, 2h, 21, 2p, and 2t are described herein as joints for convenience of explanation.

The motion information generating unit 14 performs pattern matching of the human body pattern with the distance image information of each frame. The motion information generating unit 14, for example, performs pattern matching of the human body surface of the human body pattern illustrated in FIG. 2B with the distance image illustrated in FIG. 2A, thereby extracting a person in a certain posture from the distance image information. Thus, the motion information generating unit 14 obtains the coordinates of the human body surface of the person extracted from the distance image. As described above, a relative positional relation between the human body surface and each joint in the human body pattern is known. The motion information generating unit 14 calculates the coordinates of the respective joints in the person from the coordinates of the human body surface of the person extracted from the distance image. As illustrated in FIG. 2C, the motion information generating unit 14 obtains the coordinates of the respective joints forming the skeletal structure of the human body from the distance image information. The obtained coordinates of the respective joints are coordinates in the distance coordinate system.

In the pattern matching, the motion information generating unit 14 may supplementarily use information indicating the positional relation of the joints. The information indicating the positional relation of the joints includes connection relations between joints (e.g., “the joint 2a and the joint 2b are connected”) and ranges of motion of the respective joints, for example. A joint is a part connecting two or more bones. An angle formed by bones changes in association with a change in posture, and the range of motion varies depending on the joints. The range of motion is represented by the maximum value and the minimum value of the angle formed by bones connected by a joint, for example. The motion information generating unit 14 also learns the ranges of motion of the respective joints in the learning of the human body pattern, for example. The motion information generating unit 14 stores therein the ranges of motion in association with the respective joints.

The motion information generating unit 14 converts the coordinates of the respective joints in the distance image coordinate system into values represented by the world coordinate system. The world coordinate system is a coordinate system of a three-dimensional space where rehabilitation is performed. In the world coordinate system, the position of the motion information acquiring unit 10 is set as an origin, the horizontal direction corresponds to an x-axis, the vertical direction corresponds to a y-axis, and a direction orthogonal to the xy-plane corresponds to a z-axis, for example. The value of the coordinates in the z-axis direction may be referred to as a “depth”.

The following describes the conversion processing from the distance image coordinate system to the world coordinate system. In the first embodiment, the motion information generating unit 14 stores therein in advance a conversion equation used for conversion from the distance image coordinate system to the world coordinate system. The conversion equation receives coordinates in the distance image coordinate system and an incident angle of reflected light corresponding to the coordinates and outputs coordinates in the world coordinate system, for example. The motion information generating unit 14, for example, inputs coordinates (X1, Y1, Z1) of a certain joint and an incident angle of reflected light corresponding to the coordinates to the conversion equation, thereby converting the coordinates (X1, Y1, Z1) of the certain joint into coordinates (x1, y1, z1) in the world coordinate system. Because the correspondence relation between the coordinates in the distance image coordinate system and the incident angle of reflected light is known, the motion information generating unit 14 can input the incident angle corresponding to the coordinates (X1, Y1, Z1) to the conversion equation. The explanation has been made of the case where the motion information generating unit 14 converts the coordinates in the distance image coordinate system into the coordinates in the world coordinate system. Alternatively, the motion information generating unit 14 can convert the coordinates in the world coordinate system into the coordinates in the distance image coordinate system.

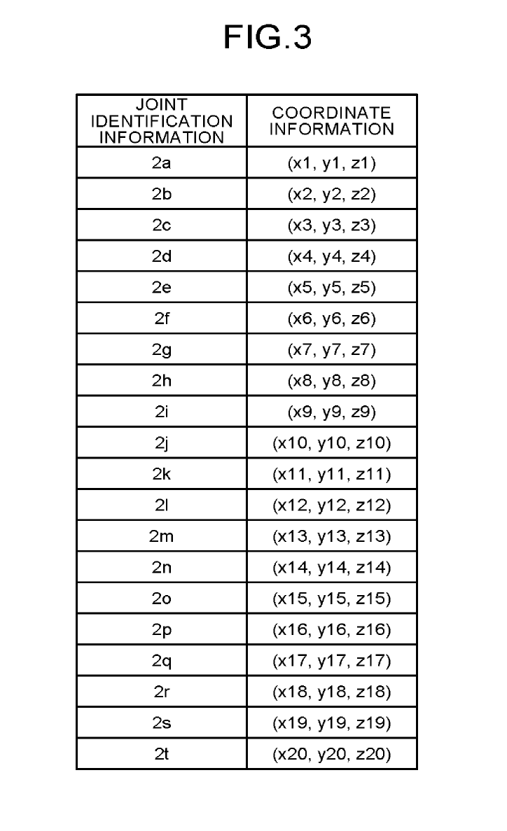

The motion information generating unit 14 generates skeletal information from the coordinates of the respective joints represented by the world coordinate system. FIG. 3 is a diagram of an example of the skeletal information generated by the motion information generating unit 14. The skeletal information of each frame includes capturing time information of the frame and the coordinates of the respective joints. As illustrated in FIG. 3, the motion information generating unit 14 generates skeletal information in which joint identification information is associated with coordinate information, for example. In FIG. 3, the capturing time information is not illustrated. The joint identification information is identification information used to identify a joint and is set in advance. Joint identification information “2a” corresponds to the head, and joint identification information “2b” corresponds to the intermediate portion between the shoulders, for example. The other pieces of joint identification information similarly indicate respective joints corresponding thereto. The coordinate information indicates the coordinates of the respective joints in each frame in the world coordinate system.

In the first row of FIG. 3, the joint identification information “2a” is associated with coordinate information “(x1, y1, z1)”. In other words, the skeletal information listed in FIG. 3 indicates that the head is present at the position of the coordinates (x1, y1, z1) in a certain frame. In the second row of FIG. 3, the joint identification information “2b” is associated with coordinate information “(x2, y2, z2)”. In other words, the skeletal information listed in FIG. 3 indicates that the intermediate portion between the shoulders is present at the position of the coordinates (x2, y2, z2) in the certain frame. The other pieces of joint identification information similarly indicate that the joints are present at the positions of the respective coordinates in the certain frame.

Every time the motion information generating unit 14 receives the distance image information of each frame from the distance image acquiring unit 12, the motion information generating unit 14 performs pattern matching on the distance image information of each frame. The motion information generating unit 14 thus performs conversion from the distance image coordinate system to the world coordinate system, thereby generating the skeletal information of each frame. The motion information generating unit 14 then outputs the generated skeletal information of each frame to the motion information processing apparatus 100 and stores the skeletal information in a motion information storage unit, which will be described later.

The processing of the motion information generating unit 14 is not necessarily performed by the method described above. While the explanation has been made of the method in which the motion information generating unit 14 uses a human body pattern to perform pattern matching, the embodiment is not limited thereto. Instead of the human body pattern or in addition to the human body pattern, the motion information generating unit 14 may use a pattern of each part to perform pattern matching.

While the explanation has been made of the method in which the motion information generating unit 14 obtains the coordinates of the respective joints from the distance image information in the description above, for example, the present embodiment is not limited thereto. The motion information generating unit 14 may obtain the coordinates of respective joints using color image information in addition to the distance image information, for example. In this case, the motion information generating unit 14, for example, performs pattern matching of a human body pattern represented by a color image coordinate system with the color image information, thereby obtaining the coordinates of the human body surface from the color image information. The color image coordinate system has no information on “distance Z” included in the distance image coordinate system. The motion information generating unit 14 acquires the information on “distance Z” from the distance image information, for example. The motion information generating unit 14 then performs arithmetic processing using the two pieces of information, thereby obtaining the coordinates of the respective joints in the world coordinate system.

The motion information generating unit 14 outputs the color image information generated by the color image acquiring unit 11, the distance image information generated by the distance image acquiring unit 12, and the audio recognition result output from the audio recognizing unit 13 to the motion information processing apparatus 100 as needed. The motion information generating unit 14 then stores the pieces of information in the motion information storage unit, which will be described later. Pixel positions in the color image information can be associated with pixel positions in the distance image information in advance based on the positions of the color image acquiring unit 11 and the distance image acquiring unit 12 and the capturing direction. As a result, the pixel positions in the color image information and the pixel positions in the distance image information can also be associated with the world coordinate system derived by the motion information generating unit 14. The association processing and the use of the distance (m) calculated by the distance image acquiring unit 12 makes it possible to calculate the height and the length of each part of the body (the length of the arm and the length of the abdomen) and to calculate the distance between two pixels specified on a color image. Similarly, the capturing time information of the color image information can be associated with the capturing time information of the distance image information in advance. The motion information generating unit 14 refers to the audio recognition result and the distance image information. If the joint 2a is present near the direction in which a word recognized as audio at certain time is spoken, the motion information generating unit 14 can output the word as a word spoken by a person having the joint 2a. The motion information generating unit 14 outputs the information indicating the positional relation of the joints to the motion information processing apparatus 100 as needed and stores the information in the motion information storage unit, which will be described later.

While the explanation has been made of the case where the motion information acquiring unit 10 detects a motion of one person, the embodiment is not limited thereto. If a plurality of persons is included in the capturing range of the motion information acquiring unit 10, the motion information acquiring unit 10 may detect motions of the persons. If a plurality of persons is captured in the distance image information of a single frame, the motion information acquiring unit 10 associates pieces of skeletal information on the persons generated from the distance image information of the single frame with one another. The motion information acquiring unit 10 then outputs the skeletal information to the motion information processing apparatus 100 as the motion information.

The configuration of the motion information acquiring unit 10 is not limited to the configuration described above. In the case where the motion information is generated by detecting a motion of a person with another motion capture, such as an optical, a mechanical, or a magnetic motion capture, for example, the motion information acquiring unit 10 does not necessarily include the distance image acquiring unit 12. In this case, the motion information acquiring unit 10 includes markers attached to the human body so as to detect a motion of the person and a sensor that detects the markers as a motion sensor. The motion information acquiring unit 10 detects a motion of the person with the motion sensor, thereby generating the motion information. The motion information acquiring unit 10 uses the positions of the markers included in an image captured by the color image acquiring unit 11 to associate the pixel positions in the color image information with the coordinates in the motion information. The motion information acquiring unit 10 outputs the motion information to the motion information processing apparatus 100 as needed. In the case where the motion information acquiring unit 10 outputs no audio recognition result to the motion information processing apparatus 100, for example, the motion information acquiring unit 10 does not necessarily include the audio recognizing unit 13.

While the motion information acquiring unit 10 outputs the coordinates in the world coordinate system as the skeletal information in the embodiment, the embodiment is not limited thereto. The motion information acquiring unit 10 may output the coordinates in the distance image coordinate system yet to be converted, for example. The conversion from the distance image coordinate system to the world coordinate system may be performed by the motion information processing apparatus 100 as needed.

Referring back to FIG. 1, the motion information processing apparatus 100 uses the motion information output from the motion information acquiring unit 10 to perform processing for supporting rehabilitation. The motion information processing apparatus 100 is an information processing apparatus, such as a computer and a workstation. As illustrated in FIG. 1, the motion information processing apparatus 100 includes an output unit 110, an input unit 120, a storage unit 130, and a control unit 140.

The output unit 110 outputs various types of information used to support rehabilitation. The output unit 110, for example, displays a graphical user interface (GUI) used by an operator who operates the motion information processing apparatus 100 to input various types of requests with the input unit 120, displays an output image generated by the motion information processing apparatus 100, or outputs a warning sound. The output unit 110 is a monitor, a speaker, headphones, or a headphone part of a headset, for example. The output unit 110 may be a display attached to a body of a user, such as a glasses-like display and a head mount display.

The input unit 120 receives input of various types of information used to support rehabilitation. The input unit 120, for example, receives input of various types of requests from the operator of the motion information processing apparatus 100 and transfers the various types of received requests to the motion information processing apparatus 100. The input unit 120 is a mouse, a keyboard, a touch command screen, a trackball, a microphone, or a microphone part of a headset, for example. The input unit 120 may be a sensor that acquires biological information, such as a sphygmomanometer, a heart rate meter, and a thermometer.

The storage unit 130 is a semiconductor memory element, such as a random access memory (RAM) and a flash memory, or a storage device, such as a hard disk device and an optical disk device, for example. The control unit 140 is provided by an integrated circuit, such as an application specific integrated circuit (ASIC) and a field programmable gate array (FPGA), or a central processing unit (CPU) executing a predetermined computer program.

The configuration of the motion information processing apparatus 100 according to the first embodiment has been described. With this configuration, the motion information processing apparatus 100 according to the first embodiment analyzes the motion information on a person acquired by the motion information acquiring unit 10, thereby supporting rehab and improving the quality of the rehab. Specifically, the motion information processing apparatus 100 according to the first embodiment includes an acquiring unit that acquires motion information indicating a motion of a person and an output unit that outputs support information used to support a motion relating to rehab for the person whose motion information is acquired by the acquiring unit. Thus, the motion information processing apparatus 100 according to the first embodiment improves the quality of the rehab.

The motion information processing apparatus 100 according to the present application acquires the motion information on a person who engages in rehab and outputs the support information to the person. Persons who engage in rehab include an object person serving as a target of rehab and an assistant who assists the object person. The first embodiment to a fourth embodiment describe a case where support is provided for an object person, whereas a fifth embodiment to a ninth embodiment describe a case where support is provided for an assistant.

The motion information processing apparatus 100 according to the first embodiment having the configuration described above provides support for an object person. Specifically, the motion information processing apparatus 100 according to the first embodiment analyzes the motion information on an object person who performs rehab, which motion information is acquired by the motion information acquiring unit 10, thereby supporting the rehab of the object person.

The motion information processing apparatus 100 according to the present embodiment enables an object person to perform effective rehab without physical support by the processing described below in detail. In exercise therapy of rehab these days, movement training, gait training, range of joint motion exercises, muscle building training, and the like are performed with the support of assistants, such as physical therapists and care workers. In such exercise therapy, a training menu is determined based on appropriate instructions from a rehabilitation specialist, for example. An assistant, such as a physical therapist and a care worker, urges an object person to perform the determined training menu while issuing instructions next to the object person.

In such a training menu for rehab, regulations (rules) may be set for each type of training. In stair-climbing training of gait training performed by an object person having a disability in one foot, for example, the following rule is set: “the object person steps forward with the foot having no disability when going up stairs and steps forward with the foot having the disability when going down stairs”. In a range of joint motion exercise performed by an object person having a disability in the arms, for example, the following rule is set: “the object person raises the arms to shoulder level and rotates the wrists”. Such rules can be observed when rehab is performed while an assistant, such as a physical therapist and a care worker, is drawing the object person's attention next to the object person.

When the object person performs the training menu alone, however, such rules may not possibly be observed. In recent years, a severe shortage of assistants who supports rehab has been pointed out. There is an increasing demand for a rehab supporting method for enabling an object person to perform rehab correctly and effectively without physical support for the rehab. The motion information processing apparatus 100 according to the first embodiment performs processing using the motion information acquired by the motion information acquiring unit 10. Thus, the motion information processing apparatus 100 enables the object person to perform effective rehab without physical support.

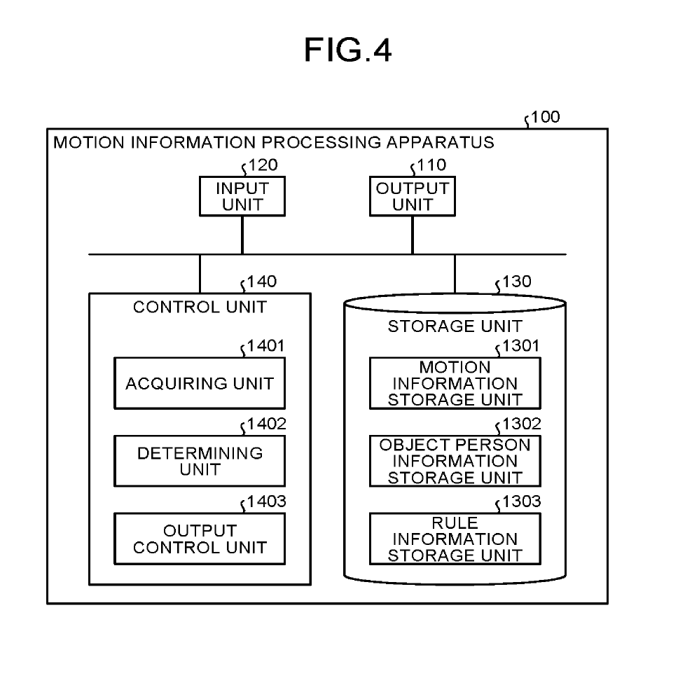

FIG. 4 is a block diagram of a detailed exemplary configuration of the motion information processing apparatus according to the first embodiment. As illustrated in FIG. 4, the storage unit 130 in the motion information processing apparatus 100 includes a motion information storage unit 1301, an object person information storage unit 1302, and a rule information storage unit 1303, for example.

The motion information storage unit 1301 stores therein various types of information acquired by the motion information acquiring unit 10. Specifically, the motion information storage unit 1301 stores therein the motion information generated by the motion information generating unit 14. More specifically, the motion information storage unit 1301 stores therein the skeletal information of each frame generated by the motion information generating unit 14. The motion information storage unit 1301 can also store therein the color image information, the distance image information, and the audio recognition result of each frame output from the motion information generating unit 14 in a manner associated with one another.

The object person information storage unit 1302 stores therein various types of information on the object person who performs rehab. Specifically, the object person information storage unit 1302 stores therein object person information including examination data and information on a disease part of the object person, for example. The object person information stored in the object person information storage unit 1302 is acquired from a medical information system, a personal health record (PHR), or the like. The medical information system is an information system used in hospital. Examples of the medical information system include electronic chart systems, receipt computer processing systems, ordering systems, reception (personal or qualification authentication) systems, and diagnosis support systems. The PHR is a record obtained by collecting and managing medical information, healthcare information, and health information scattered in medical institutions, medical examination institutions, sports gyms, and home, for example. The PHR is managed mainly by an individual with a management system built on a network, for example.

In the case where the motion information processing apparatus 100 is connected to the medical information system via a network, for example, the control unit 140 receives an acquisition request of object person information from the operator of the motion information processing apparatus 100 via the input unit 120. The control unit 140 then acquires the object person information from the medical information system and stores the acquired object person information in the object person information storage unit 1302. The input unit 120 receives information on a name, a name number, or the like of the object person as the acquisition request of the object person information.

By contrast, in the case where the motion information processing apparatus 100 is not connected to the medical information system via a network, the operator can use a portable storage medium, such as an external hard disk, a flash memory, a memory card, a flexible disk (FD), a compact disc read only memory (CD-ROM), a magnetic optical disc (MO), and a digital versatile disc (DVD), to move the object person information from the medical information system to the motion information processing apparatus 100. Alternatively, the operator may not move the object person information to the motion information processing apparatus 100 but use the portable storage medium in a manner connected to the motion information processing apparatus 100 as the object person information storage unit 1302. In the case where the motion information processing apparatus 100 is connected to the medical information system via a network, the operator can use the portable storage medium to move the object person information from the medical information system to the motion information processing apparatus 100. An example of the object person information will be described.

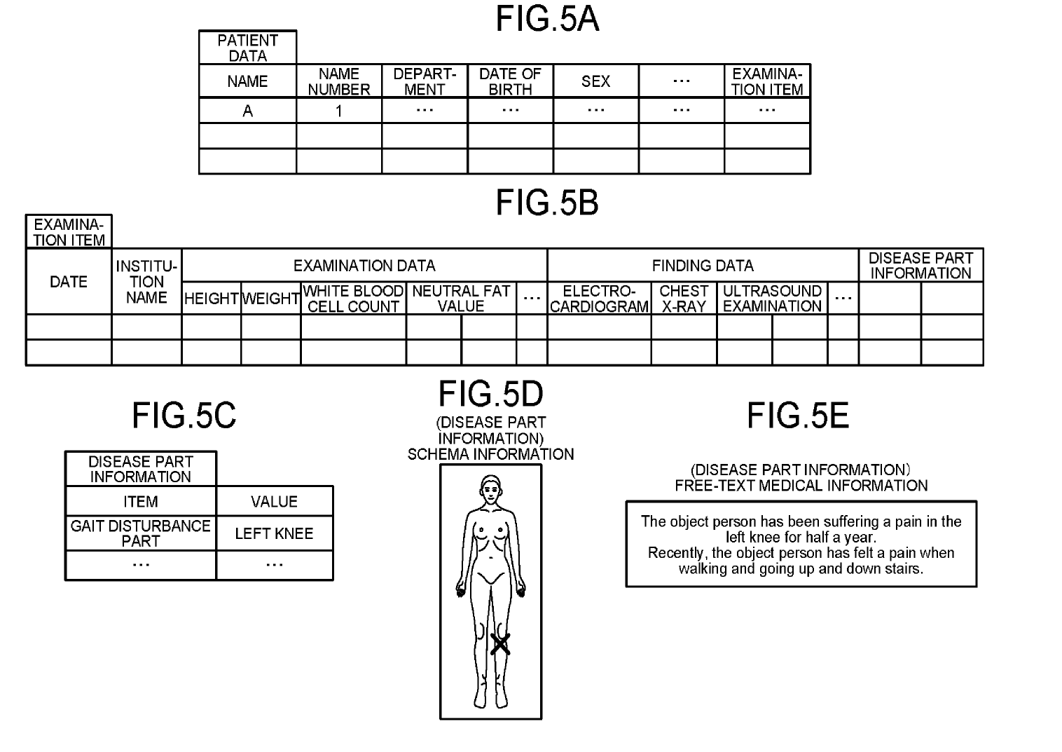

FIG. 5A to FIG. 5E are diagrams of an example of the object person information stored in the object person information storage unit 1302 according to the first embodiment. FIG. 5A to FIG. 5E illustrate an example of structured object person information. Specifically, FIG. 5A illustrates an example of patient data stored for each object person. FIG. 5B illustrates an example of examination items included in the patient data of each object person illustrated in FIG. 5A. FIGS. 5C to 5E each illustrate an example of disease part information included in the examination items illustrated in FIG. 5B.

As illustrated in FIG. 5A, for example, the object person information storage unit 1302 stores therein patient data in which a name, a name number, a department, a date of birth, a sex, and an examination item are associated with one another for each object person. The patient data illustrated in FIG. 5A is information used to identify an object person. The “name” indicates the name of the object person. The “name number” indicates an identifier used to uniquely identify the object person. The “department” indicates a department of the object person. The “date of birth” indicates the date of birth of the object person. The “sex” indicates the sex of the object person. The “examination item” is a space in which an item of examination taken by the object person is recorded.

As illustrated in FIG. 5B, for example, the object person information storage unit 1302 stores therein examination items in which a date, an institution name, examination data, finding data, and disease part information are associated with one another. The “date” illustrated in FIG. 5B indicates a data on which the object person takes an examination. The “institution name” indicates a name of a medical institution at which the object person takes an examination. The “examination data” indicates numerical data of an examination taken by the object person. The “finding data” indicates findings of a doctor about an examination taken by the object person. The “disease part information” indicates information on a part of a disability in the object person.

As illustrated in FIG. 5B, the “examination data” includes a height, a weight, a white blood cell count, and a neutral fat value, for example. Numerical values of examination results are recorded in respective items. As illustrated in FIG. 5B, the “finding data” includes an electrocardiogram, a chest X-ray, and an ultrasound examination, for example. Finding data, such as “normal”, “evaluation A”, and “evaluation B”, are recorded in respective items.

The disease part information illustrated in FIG. 5B includes disease part information illustrated in FIGS. 5C to 5E, for example. The disease part information includes disease part information structured by associating items with values as illustrated in FIG. 5C, for example. The “item” indicates for what kind of action the object person is disabled. The “value” indicates a disease part in a body. Information on “item: gait disturbance part, value: left knee” illustrated in FIG. 5C indicates that the left knee is a disease part for a gait, for example.

As illustrated in FIG. 5D, schema information is included as the disease part information, for example. As illustrated in FIG. 5D, the disease part information includes schema information in which a mark is placed on the left knee of a schema of a whole human body, for example.

As illustrated in FIG. 5E, free-text medical information is included as the disease part information, for example. As illustrated in FIG. 5E, the disease part information includes free-text medical information like a comment written in a comment column of a chart that “the object person has been suffering a pain in the left knee for half a year. Recently, the object person has felt a pain when walking and going up and down stairs”, for example.

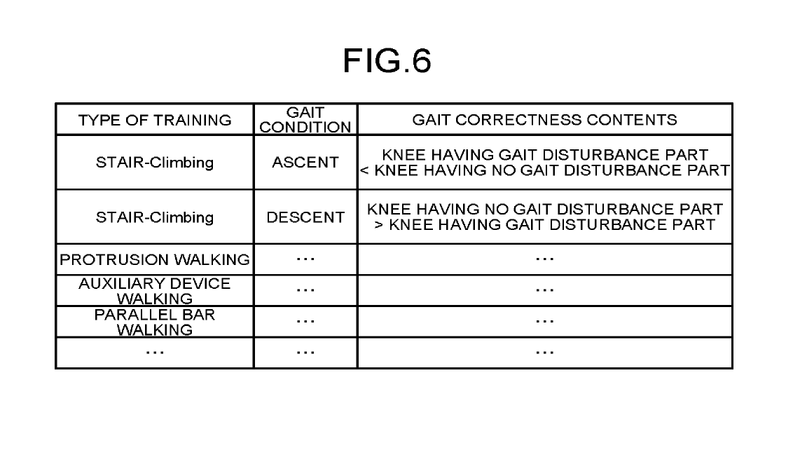

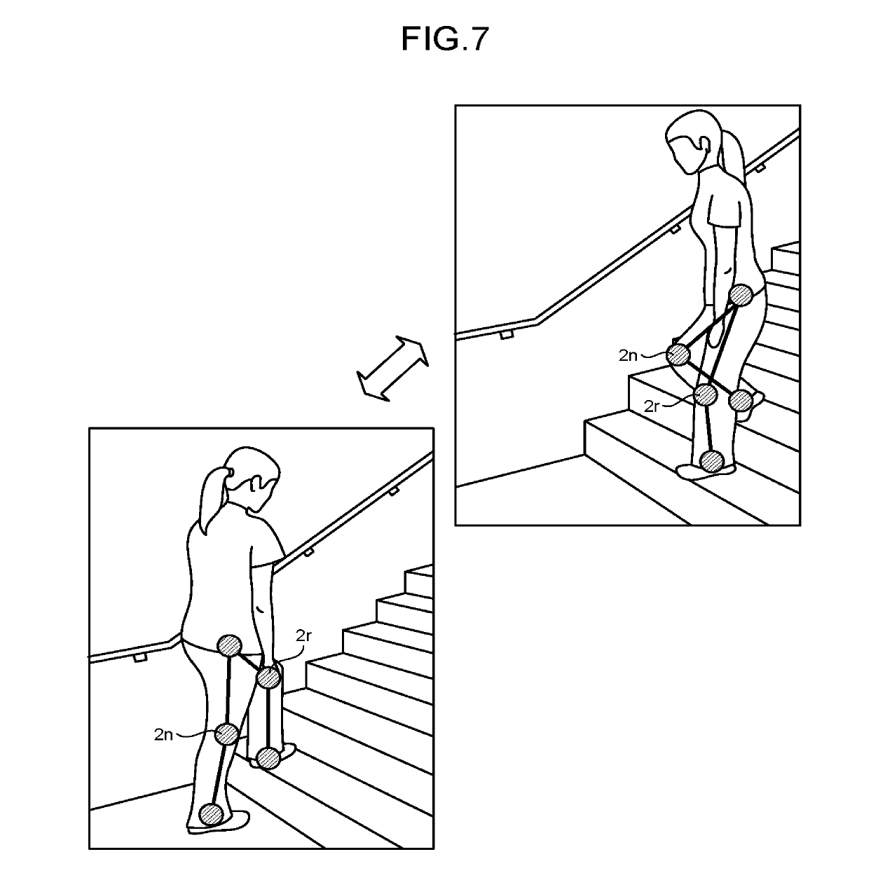

Referring back to FIG. 4, the rule information storage unit 1303 stores therein rule information on the object person in rehabilitation. Specifically, the rule information storage unit 1303 stores therein rule information serving as information on regulations (rules) set for each type of training in rehabilitation. FIG. 6 is a diagram of an example of the rule information stored in the rule information storage unit 1303 according to the first embodiment. FIG. 6 illustrates rule information in which rules are associated with respective types of training in gait training.

As illustrated in FIG. 6, the rule information storage unit 1303 stores therein rule information in which a type of training, a gait condition, and gait correctness contents are associated with one another, for example. As illustrated in FIG. 6, for example, the rule information storage unit 1303 stores therein rule information on “type of training: stair-climbing, gait condition: ascent, gait correctness contents: the knee having a gait disturbance part<the knee having no gait disturbance part”. The information indicates that “the knee having a gait disturbance part” is kept from being higher than “the knee having no gait disturbance part” in “ascent” in the training of “stair-climbing”. In other words, if “the knee having a gait disturbance part” is higher than “the knee having no gait disturbance part”, the walking is not correct.

This is rule information set based on the following fact: if the object person always steps forward with “the knee having no gait disturbance part” when going up stairs, “the knee having gait disturbance part” cannot be higher than “the knee having no gait disturbance part”. In other words, if the object person goes up stairs in a manner keeping “the knee having gait disturbance part” from being higher than “the knee having no gait disturbance part”, the object person always steps forward with “the knee having no gait disturbance part”.

Similarly, as illustrated in FIG. 6, the rule information storage unit 1303 stores therein rule information on “type of training: stair-climbing, gait condition: descent, gait correctness contents: the knee having no gait disturbance part>the knee having a gait disturbance part”. The information indicates that “the knee having no gait disturbance part” is kept from being lower than “the knee having a gait disturbance part” in “descent” in the training of “stair-climbing”. In other words, if “the knee having no gait disturbance part” is lower than “the knee having a gait disturbance part”, the walking is not correct.

The rule information illustrated in FIG. 6 is given by way of example of gait training. In other words, the rule information storage unit 1303 stores therein various types of rule information for respective types of training, such as movement training, range of joint motion exercises, and muscle building training. The rule information storage unit 1303, for example, stores therein rule information on “type of training: a range of joint motion of the upper limbs, target condition: the whole arms, correctness contents: the level of the shoulder joints=the level of the elbow joints and rotation of the wrists” as rule information on “the object person raises the arms to shoulder level and rotates the wrists”, which is a rule of a range of joint motion exercise performed by the object person having a disability in the arms. The information indicates that “rotation of the wrists” is performed making “the level of the elbow joints” nearly the same as “the level of the shoulder joints” for “the whole arms” in the training of “a range of joint motion of the upper limbs”. In other words, if “rotation of the wrists” is performed in a state where “the level of the elbow joints” is yet to reach “the level of the shoulder joints”, the range of joint motion exercise is not correct.

As described above, the rule information storage unit 1303 stores therein various types of rule information for each type of training. The rule information may be acquired via the network in the same manner as the object person information. Alternatively, the rule information may be directly input by the operator through the input unit 120. In the rule information, unique rules may be set for each hospital or each assistant.

Referring back to FIG. 4, the control unit 140 in the motion information processing apparatus 100 includes an acquiring unit 1401, a determining unit 1402, and an output control unit 1403, for example. The motion information processing apparatus 100 uses various types of information stored in the storage unit 130, thereby enabling an object person to perform effective rehab without physical support. While the following describes an example in which stair-climbing training is performed as rehab, the embodiment is not limited thereto.

The acquiring unit 1401 acquires the motion information on the object person serving as a target of rehabilitation. Specifically, the acquiring unit 1401 acquires the motion information acquired by the motion information acquiring unit 10 and stored in the motion information storage unit 1301. More specifically, the acquiring unit 1401 acquires the skeletal information of each frame stored by the motion information storage unit 1301.

The acquiring unit 1401, for example, acquires skeletal information subsequent to execution of a motion corresponding to the contents of rehabilitation. The acquiring unit 1401 acquires skeletal information of each frame obtained after the object person who performs the stair-climbing training ascends a step of the stairs, for example. In other words, the acquiring unit 1401 acquires the skeletal information from a frame obtained when the object person who goes up the stairs starts the motion to a frame obtained after the object person ascends a step of the stairs, which skeletal information is acquired by the motion information acquiring unit 10.

The determining unit 1402 determines whether a motion of the object person indicated by the motion information acquired by the acquiring unit 1401 follows the regulations included in the rule information based on the rule information on the object person in rehabilitation. Specifically, the determining unit 1402 determines whether a motion of the object person indicated by the motion information follows the regulations included in the rule information based on the rule information determined by the contents of rehabilitation performed by the object person and the information on an affected area of the object person. The determining unit 1402, for example, determines whether a motion indicated by the motion information subsequent to execution of the motion acquired by the acquiring unit 1401 follows the rules included in the rule information.

The determining unit 1402, for example, acquires the object person information on the object person who performs rehab from the object person information stored in the object person information storage unit 1302. The determining unit 1402 then extracts a disease part of the object person from the disease part information included in the acquired object person information. If the determining unit 1402 receives information that an object person whose patient data is “name: A, name number: 1” performs stair-climbing training via the input unit 120, for example, the determining unit 1402 refers to the examination items included in the corresponding patient data, thereby extracting the object person's “disease part: left knee” (refer to FIG. 5). The determining unit 1402 extracts the disease part using the “item” in the disease part information as a key, extracts the disease part based on the position (e.g., a “cross”) of the information depicted on the schema, or extracts the disease part from the free text using a text mining technology, for example.

The determining unit 1402 then refers to the rule information stored in the rule information storage unit 1303, thereby extracting the rules in stair-climbing training (refer to FIG. 6). The determining unit 1402, for example, refers to the rule information illustrated in FIG. 6, thereby acquiring the rule of “the knee having a gait disturbance part<the knee having no gait disturbance part” of the “gait condition: ascent” in the “type of training” of “stair-climbing” and the rule of “the knee having no gait disturbance part>the knee having a gait disturbance part” of the “gait condition: descent”. Subsequently, the determining unit 1402 determines whether the stair-climbing training of the object person “name: A” having a disability in the “left knee” is performed in accordance with the rules based on the motion information on the object person “name: A” acquired by the acquiring unit 1401.

FIG. 7 is a view for explaining an example of the determination processing performed by the determining unit 1402 according to the first embodiment. FIG. 7 schematically illustrates the case where the determining unit 1402 determines whether the stair-climbing training of the object person “name: A” having a disability in the “left knee” is performed in accordance with the rules. FIG. 7 illustrates schematics obtained by superimposing the color image information acquired by the motion information acquiring unit 10 using the object person “name: A” going up and down the stairs as the photographic subject and a part of the skeletal information generated based on the distance image information.

The determining unit 1402, for example, determines that the object person “name: A” has a disability in the “left knee” from the object person information. The determining unit 1402 sets the following determination criterion for the object person “name: A” based on the rule information: the object person “name: A” steps forward with the right foot when going up the stairs and steps forward with the left foot when going down the stairs. The determining unit 1402 determines whether the motion of the object person “name: A” indicated by the motion information (skeletal information) acquired from the motion information storage unit 1301 by the acquiring unit 1401 satisfies the determination criterion.

In other words, the determining unit 1402 refers to coordinate information of the joint identification information “2n” corresponding to the right knee and coordinate information of the joint identification information “2r” corresponding to the left knee in the skeletal information acquired for each frame as illustrated in FIG. 7. The determining unit 1402 determines whether the left knee is higher than the right knee when the object person “name: A” is going up the stairs and determines whether the right knee is lower than the left knee when the object person “name: A” is going down the stairs. Thus, the determining unit 1402 determines whether the motion of the object person “name: A” satisfies the determination criterion.

In other words, the determining unit 1402 compares a value “y14” in the y-coordinate of the joint identification information “2n” corresponding to the right knee with a value “y18” in the y-coordinate of the joint identification information “2r” corresponding to the left knee in each frame. The determining unit 1402 determines whether “y14>y18” is satisfied (refer to FIG. 3). If “y14<y18” is satisfied, the determining unit 1402 determines that the rehab being performed is not performed in accordance with the rules. In this case, the determining unit 1402 outputs a determination result indicating that the rehab is not performed in accordance with the rules to a notifying unit.

In the stair-climbing illustrated in the left figure of FIG. 7, for example, the left knee is higher than the right knee when the object person “name: A” is going up the stairs (the object person “name: A” steps forward with the left foot having the disability). The determining unit 1402 determines that the rehab does not follow the rules. By contrast, in the stair-climbing illustrated in the right figure of FIG. 7, the right knee is not lower than the left knee when the object person “name: A” is going down the stairs (the object person “name: A” steps forward with the left foot having the disability). The determining unit 1402 determines that the rehab follows the rules.

As described above, the determining unit 1402 determines in each frame whether the motion of the object person performing the rehab while successively moving follows the rules acquired for each object person with the coordinate information (x, y, z) of the skeletal information of each frame acquired by the motion information acquiring unit 10. While the explanation has been made of the case where stair-climbing training is performed in the example described above, the determining unit 1402 similarly performs determination processing on other types of training with the coordinate information (x, y, z) of the skeletal information of each frame.

When an object person having a disability in the arms performs a range of joint motion exercise, for example, the determining unit 1402 refers to the object person information in the object person information storage unit 1302, thereby acquiring the fact that the object person has a disability in the arms. If the determining unit 1402 receives an operation indicating execution of the range of joint motion exercise from the object person, the determining unit 1402 acquires the rule information on “type of training: a range of joint motion of the upper limbs, target condition: the whole arms, correctness contents: the level of the shoulder joints=the level of the elbow joints and rotation of the wrists” stored in the rule information storage unit 1303 to make determination. In other words, the determining unit 1402 compares the values of the elbow joints “2f” and “2j” in the y-coordinate with the values of the shoulder joints “2e” and “2i” in the y-coordinate, thereby determining whether “the level of the shoulder joints=the level of the elbow joints” is satisfied.

In the state where “the level of the shoulder joints=the level of the elbow joints” is satisfied, the determining unit 1402 determines whether “rotation of the wrists” is performed, that is, whether the coordinates of the hand joints “2h” and “2l” rotate with the coordinates of the wrist joints “2g” and “2k”, respectively, as a base point. If the coordinates of the right hand joint “2h” rotate in the state where the value of the right elbow joint “2f” in the y-coordinate is not nearly the same as the value of the right shoulder joint “2e” in the y-coordinate or if the coordinates of the left hand joint “21” rotate in the state where the value of the left elbow joint “2j” in the y-coordinate is not nearly the same as the value of the left shoulder joint “2i” in the y-coordinate, the determining unit 1402 determines that the exercise is not correctly performed.

While the explanation has been made of the case where only the coordinate information of the skeletal information is used in the example of determination, the embodiment is not limited thereto. A predetermined threshold may be added to the coordinate information, for example. In an example of determination in the stair-climbing, to compare the value “y14” in the y-coordinate of the joint identification information “2n” corresponding to the right knee and the value “y18” in the y-coordinate of the joint identification information “2r” corresponding to the left knee in each frame, the determining unit 1402 adds a predetermined threshold “a” to the value “y18” in the y-coordinate of “2r” and determines whether “y14>y18+a” is satisfied, for example. In other words, if “y14<y18+a” is satisfied, the determining unit 1402 determines that the rehab being performed is not performed in accordance with the rules. This enables more reliable determination of the foot with which the object person steps forward when going up and down the stairs, for example.

While the explanation has been made of the case where the determination is made based on the levels of the knees in the example of determination in the stair-climbing, the embodiment is not limited thereto. The determination may be made based on another joint of the legs, for example. The determination may be made based on the coordinate information on ankle joints, for example. In this case, the rule information stored in the rule information storage unit 1303 is a rule of “the ankle having a gait disturbance part<the ankle having no gait disturbance part” in the “gait condition: ascent” and a rule of “the ankle having no gait disturbance part>the ankle having a gait disturbance part” in the “gait condition: descent”. Alternatively, the determination may be made comprehensively using the levels of joints at two points, for example.

While the explanation has been made of the case where only the “values in the y-coordinate” are used in the example of determination of the levels of the knees and the example of determination of the levels of the shoulders and the elbows described above, the embodiment is not limited thereto. The determination may be made by taking into account at least one of the “values in the x-coordinate” and the “values in the z-coordinate”, for example. In this case, rule information taking into account each of the values is stored in the rule information storage unit 1303.

Referring back to FIG. 4, the output control unit 1403 controls the output unit 110 to output the result of determination made by the determining unit 1402. The output control unit 1403, for example, controls the output unit 110 to output light and sound, thereby notifying the object person performing the rehab of the fact that the motion does not follow the rules. The output control unit 1403, for example, causes the output unit 110 to blink with red light on a display surface or output a warning sound, thereby notifying the object person performing the rehab of the fact.

The output control unit 1403 can notify the object person of the fact with audio. If the object person steps forward with the wrong foot to go up the stairs, for example, the output control unit 1403 can notify the object person to step forward with the correct foot with audio.

As described above, when the object person is performing the rehab alone, the motion information processing apparatus 100 according to the first embodiment extracts the rules of the rehab for each object person, thereby determining whether the motion indicated by the motion information follows the rules. If the motion does not follow the rules, the motion information processing apparatus 100 notifies the object person of the determination result. As a result, the motion information processing apparatus 100 according to the first embodiment enables the object person to perform effective rehab without physical support for the object person.

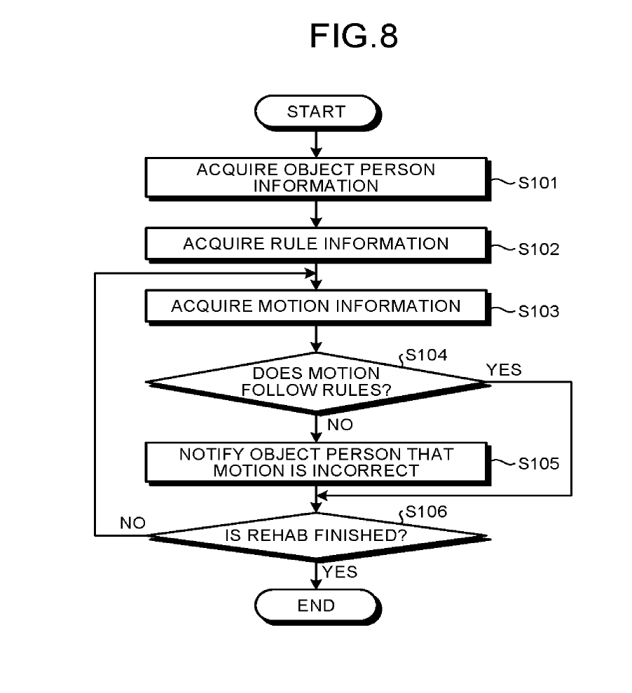

The following describes processing of the motion information processing apparatus 100 according to the first embodiment with reference to FIG. 8. FIG. 8 is a flowchart of a procedure of processing performed by the motion information processing apparatus 100 according to the first embodiment. FIG. 8 illustrates processing performed after an instruction operation to start support for rehab is carried out by an object person.

As illustrated in FIG. 8, if an instruction to start support is received in the motion information processing apparatus 100 according to the first embodiment, the determining unit 1402 acquires object person information on the object person who performs the rehab from the object person information storage unit 1302 (Step S101). The determining unit 1402 then acquires rule information corresponding to the acquired object person information from the rule information storage unit 1303 (Step S102). The acquiring unit 1401 acquires motion information (skeletal information) (Step S103).

Subsequently, the determining unit 1402 determines whether a motion of the object person indicated by the motion information follows rules included in the acquired rule information (Step S104). If it is determined that the motion follows the rules (Yes at Step S104), the determining unit 1402 determines whether the rehab is finished (Step S106).

By contrast, if it is determined that the motion does not follow the rules (No at Step S104), the output control unit 1403 notifies the object person that the motion is incorrect (Step S105). The determining unit 1402 then determines whether the rehab is finished (Step S106). If it is determined that the rehab is not finished yet at Step S106 (No at Step S106), the system control is returned to Step S103, and the acquiring unit 1401 acquires motion information. By contrast, if the rehab is finished (Yes at Step S106), the motion information processing apparatus 100 terminates the processing.

As described above, according to the first embodiment, the acquiring unit 1401 acquires the motion information relating to the skeletal structure of the object person serving as a target of the rehabilitation. Based on the rule information on the object person in the rehabilitation, the determining unit 1402 determines whether the motion of the object person indicated by the motion information acquired by the acquiring unit 1401 follows the rules included in the rule information. The output control unit 1403 outputs the result of determination made by the determining unit 1402. Thus, the motion information processing apparatus 100 according to the first embodiment can notify the object person of the mistake, thereby enabling the object person to perform effective rehab without physical support for the object person.

According to the first embodiment, the determining unit 1402 determines whether the motion of the object person indicated by the motion information follows the rules included in the rule information based on the rule information determined by the contents of the rehabilitation performed by the object person and the information on an affected area of the object person. Thus, the motion information processing apparatus 100 according to the first embodiment can set the rules so as to observe considerations of each object person, thereby enabling the object person to perform rehab suitable for the object person.

According to the first embodiment, the acquiring unit 1401 acquires motion information subsequent to execution of a motion corresponding to the contents of the rehabilitation. The determining unit 1402 determines whether a motion indicated by the motion information subsequent to execution of the motion acquired by the acquiring unit 1401 follows the rules included in the rule information. Thus, the motion information processing apparatus 100 according to the first embodiment can make determination based on the motion made by the object person.

### Second Embodiment

The first embodiment has described the case where, after the object person makes a motion of the contents of the rehab (e.g., going up and down the stairs), it is determined whether the motion follows the rules. A motion information processing apparatus 100 according to a second embodiment determines, before the object person completes a motion of the contents of the rehab, whether the motion follows the rules. In other words, the motion information processing apparatus 100 according to the second embodiment predicts a motion of the object person and issues notification if the predicted motion does not follow the rules. The motion information processing apparatus 100 according to the second embodiment is different in information stored in a rule information storage unit 1303 and determination processing performed by a determining unit 1402. The following describes mainly these points.

The rule information storage unit 1303 according to the second embodiment stores therein rule information used by the determining unit 1402 to predict a motion of an object person. The rule information storage unit 1303, for example, stores therein information used to predict a posture of the object person from a positional relation of the coordinates of the joint identification information in the skeletal information and thresholds.

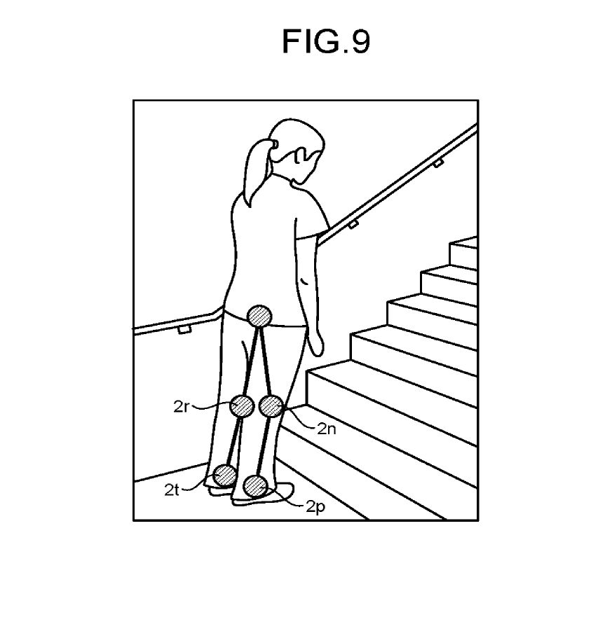

The determining unit 1402 according to the second embodiment refers to the rule information for prediction of a motion of an object person stored in the rule information storage unit 1303, thereby predicting a motion of the object person acquired by an acquiring unit 1401. FIG. 9 is a view for explaining an example of the processing performed by the determining unit 1402 according to the second embodiment. FIG. 9 illustrates a case where the determining unit 1402 predicts a motion made when the object person “name: A” having a disability in the “left knee” performs stair-climbing training.

When the object person “name: A” is about to make a motion of going up the stairs as illustrated in FIG. 9, for example, the determining unit 1402 refers to the coordinate information of the joint identification information “2p” corresponding to the tarsus of the right foot and the coordinate information of the joint identification information “2t” corresponding to the tarsus of the left foot in the skeletal information acquired for each frame. The determining unit 1402 determines that a foot corresponding to the coordinates starting to move first is the foot with which the object person steps forward and determines whether the foot with which the object person steps forward follows the rules.

If the coordinate information of the joint identification information “2t” corresponding to the tarsus of the left foot starts to move first, for example, the determining unit 1402 predicts that the left foot is the foot with which the object person steps forward. The determining unit 1402 determines that the motion does not follow the rules because the object person having a disability in the left knee steps forward with the left foot when going up the stairs. This enables an output control unit 1403 to notify the object person of the mistake before the object person actually ascends a step of the stairs. The rule information storage unit 1303 stores therein a threshold used to determine whether the coordinate information of the joint identification information “2t” corresponding to the tarsus of the left foot starts to move first (e.g., a movement distance from original coordinates). The joint used to determine whether the foot starts to move is not limited to the tarsus and may be the knee or the ankle.

The determining unit 1402 may use information on acceleration and speed to determine whether the foot starts to move. The coordinate information on each joint included in the skeletal information is acquired for each frame. This enables calculation of the acceleration and the speed of movement of each joint. The determining unit 1402, for example, calculates the acceleration in the joint identification information “2p” corresponding to the tarsus of the right foot and the acceleration in the joint identification information “2t” corresponding to the tarsus of the left foot in the skeletal information acquired for each frame. The determining unit 1402 may determine that a foot whose acceleration exceeds a predetermined threshold is the foot with which the object person steps forward and determines whether the foot with which the object person steps forward follows the rules.

The determining unit 1402 may determine the current posture of the object person based on information on the posture of the object person stored in the rule information storage unit 1303 (e.g., a positional relation of two points). The determining unit 1402 may predict what kind of motion the object person makes next, thereby determining whether the motion follows the rules.

As described above, according to the second embodiment, the acquiring unit 1401 acquires the motion information prior to execution of the motion corresponding to the contents of the rehabilitation. The determining unit 1402 determines whether a motion indicated by the motion information prior to execution of the motion acquired by the acquiring unit 1401 follows the rules included in the rule information. Thus, the motion information processing apparatus 100 according to the second embodiment can notify the object person of a mistake before the object person actually makes the motion, thereby enabling the object person to perform effective rehab without physical support for the object person.

### Third Embodiment

In the first and the second embodiments, it is determined whether the motion in the training of the rehab follows the rules. A third embodiment describes a case where a motion not directly relating to the training of rehab is determined and then the object person is notified of the determination result. A motion information processing apparatus 100 according to the third embodiment is different in information stored in a rule information storage unit 1303 and determination processing performed by a determining unit 1402 and an output control unit 1403. The following describes mainly these points.

The rule information storage unit 1303 according to the third embodiment stores therein rule information used to determine whether a motion of the object person is a motion not directly relating to the training of rehab. The rule information storage unit 1303, for example, stores therein movement of the coordinates of the joint identification information in the skeletal information obtained when the object person falls down. The rule information storage unit 1303, for example, stores therein rapid changes in the coordinates of all the pieces of joint identification information included in the skeletal information as movement of the coordinates of the joint identification information in the skeletal information obtained when the object person falls down.

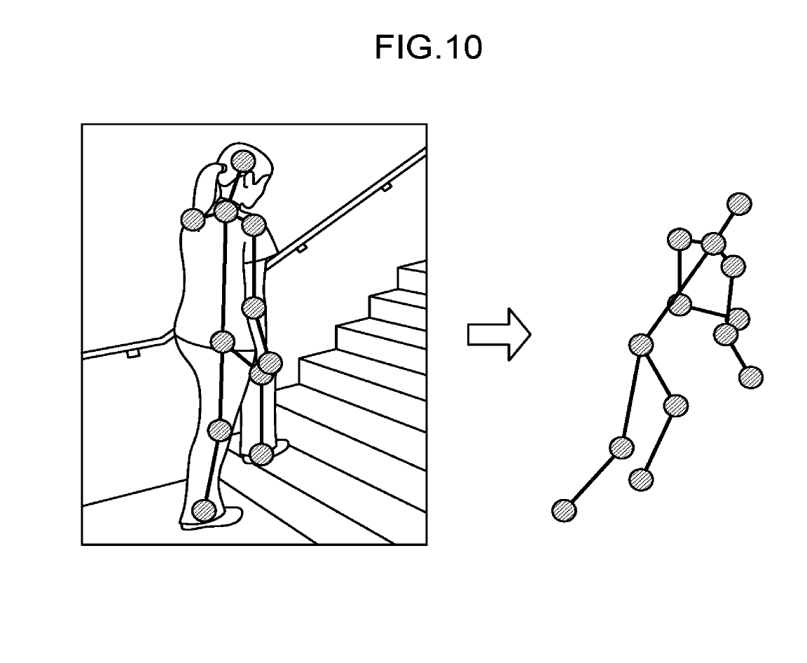

The determining unit 1402 according to the third embodiment determines whether the motion being made by the object person is a motion following the contents of the rehabilitation being currently performed based on the motion information acquired by an acquiring unit 1401. FIG. 10 is a view for explaining an example of the determination processing performed by the determining unit 1402 according to the third embodiment. If the coordinates of all the pieces of joint identification information in the skeletal information on the object person of the rehab rapidly change as illustrated in FIG. 10, for example, the determining unit 1402 determines that the object person falls down and outputs the determination result to the output control unit 1403.

If the determining unit 1402 determines that the motion being made by the object person is not a motion following the contents of the rehabilitation being currently performed, the output control unit 1403 according to the third embodiment notifies the object person of information on a motion to return to the rehabilitation. If the output control unit 1403 receives the information indicating that the object person falls down from the determining unit 1402, for example, the output control unit 1403 notifies the object person of a rule for standing up. If an object person having a disability in the left leg falls down, for example, the output control unit 1403 notifies the object person to stand up using the right leg having no disability as a pivot leg with audio.

While the embodiment has described an example in which the object person falls down, the embodiment is not limited thereto. The object person may be notified of a rule when switching the motion from going up the stairs to going down (or from going down to going up), for example. In this case, the determining unit 1402 determines a rotational motion of the whole body from movement of the coordinates of the joint identification information in the skeletal information, thereby recognizing a motion of turning around made by the object person. The determining unit 1402 determines switching of the motion from going up the stairs to going down (or from going down to going up) and outputs the result to the output control unit 1403. The output control unit 1403 notifies the object person of a rule. If an object person having a disability in the left leg switches the motion from going up to going down, for example, the output control unit 1403 notifies the object person to go down from the left foot. By contrast, If the object person switches the motion from going down to going up, the output control unit 1403 notifies the object person to go up from the right foot.

As described above, according to the third embodiment, the determining unit 1402 determines whether the motion being made by the object person is a motion following the contents of the rehabilitation being currently performed based on the motion information acquired by the acquiring unit 1401. If the determining unit 1402 determines that the motion being made by the object person is not a motion following the contents of the rehabilitation being currently performed, the output control unit 1403 notifies the object person of information on a motion to return to the rehabilitation. Thus, the motion information processing apparatus 100 according to the third embodiment can constantly determines the motion of the object person during the rehab, thereby guiding the object person to make the optimum motion.

### Fourth Embodiment

While the explanations have been made of the first to the third embodiments, the embodiments may be implemented in various different forms other than the foregoing first to the third embodiments.

The first to the third embodiments have described the stair-climbing training and the range of joint motion exercise as examples of rehab. The embodiment is not limited thereto, and muscle building training may be performed, for example. In this case, a rule information storage unit 1303 stores therein rule information corresponding to each of the training. A determining unit 1402 acquires rule information corresponding to the object person based on a disease part of the object person, thereby determining whether a motion corresponding to the motion information on the object person follows the rules.

While the first to the third embodiments have described the case where whether the training is correctly performed is determined based on the coordinates of the object person, the embodiment is not limited thereto. Whether the training is correctly performed may be determined based on the coordinates of an object, such as a bed and a wheelchair. In transfer training from a wheelchair to a bed, for example, an object person moves the wheelchair closer to the bed at a right angle with a space for lifting the legs left between the wheelchair and the bed. The object person applies the stopper of the wheelchair, lifts both legs on the bed, and brings the wheelchair into contact with the bed. The object person then moves forward until the buttocks are surely placed on the bed while doing push-up (pushing the bed surface to lift the body). Subsequently, the object person changes the direction of the body such that the head is positioned in the direction of a pillow.

In the transfer training from the wheelchair to the bed, for example, the rule information storage unit 1303 stores the space formed between the wheelchair and the bed when the object person moves the wheelchair to the bed at a right angle first depending on the body size of the object person. The rule information storage unit 1303, for example, stores therein rule information on “type of training: transfer training, target condition: from a wheelchair to a bed, correctness contents: (height: 140-150 cm, distance between objects: 30 cm), (height: 150-160 cm, distance between objects: 40 cm), . . . ”. The information indicates that a distance between objects (between a wheelchair and a bed) for each height is set in the case where “from a wheelchair to a bed” is a target in the training of “transfer”. In other words, the information indicates how much distance between the wheelchair and the bed is the optimum for each height of the object person. The distance can be optionally set and have a predetermined range.

The determining unit 1402 reads the height of the object person from the object person information and acquires a distance corresponding to the read height from the rule information storage unit 1303. The determining unit 1402 then calculates the distance between the wheelchair and the bed in each frame from the color image information acquired by the motion information acquiring unit 10. At a point when the distance between the wheelchair and the bed stops changing, the determining unit 1402 determines whether the distance at the point is the distance corresponding to the height of the object person. In the case where the height of the object person is “155 cm”, for example, the determining unit 1402 determines whether the distance between the wheelchair and the bed falls within “±5 cm” from “40 cm”. If the distance at the point when the distance between the wheelchair and the bed stops changing does not fall within the range described above, the determining unit 1402 determines that the distance is not the optimum distance for transfer and outputs the determination result to the output control unit 1403.

The distance between the wheelchair and the bed derived from the color image information can be calculated by detecting the coordinates of the wheelchair and the bed by pattern matching and using the detected coordinates, for example.

While the first to the third embodiments have described an example in which, if the determining unit 1402 determines that the motion of the object person does not follow the rules, the determining unit 1402 outputs the determination result to the output control unit 1403, and the output control unit 1403 notifies the object person of the determination result, the embodiment is not limited thereto. If the determining unit 1402 determines that the motion of the object person follows the rules, the determining unit 1402 may output the determination result to the output control unit 1403, and the output control unit 1403 may notify the object person of the determination result.

As described above, according to the first to the fourth embodiments, the motion information processing apparatus and the method according to the embodiments enables the object person to perform effective rehab without physical support.

### Fifth Embodiment

The first to the fourth embodiments have described the case where the quality of rehabilitation is improved by enabling the object person to perform effective rehab without physical support. The rehabilitation is not necessarily performed only by an object person serving as a target of the rehab. The object person may perform the rehab with assistance given by an assistant, for example. The fifth to the ninth embodiments will describe a motion information processing apparatus and a method that can increase the quality of assistance given by an assistant who assists an object person serving as a target of rehabilitation.

FIG. 11 is a schematic of an example of a distance image captured by a distance image acquiring unit 12. FIG. 11 illustrates a case where a person 4a (object person) performs rehab with the assistance of a person 4b (assistant). FIG. 11 illustrates a distance image represented by the gray scales of colors corresponding to distances with lines for convenience of explanation.

As illustrated in FIG. 11, the person 4a (object person) is performing gait training while being supported on the left arm by the right hand of the person 4b (assistant). The rehab may be performed with the assistance of the assistant in this manner.

The quality of assistance given by the assistant, however, may not possibly be maintained. Because of the relative decrease in skilled assistants caused by the recent increase in object persons, for example, the quality of assistance may not possibly be maintained. Also in the case where rehab is performed in an environment with no professional assistant, such as home and offices, the quality of assistance may not possibly be maintained, for example. A motion information processing apparatus 100a according to the fifth embodiment can increase the quality of assistance given by an assistant by the processing described below.

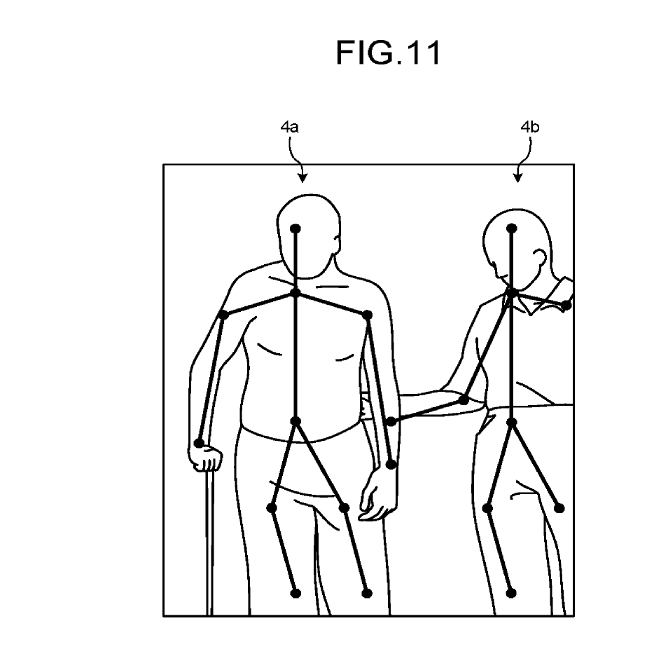

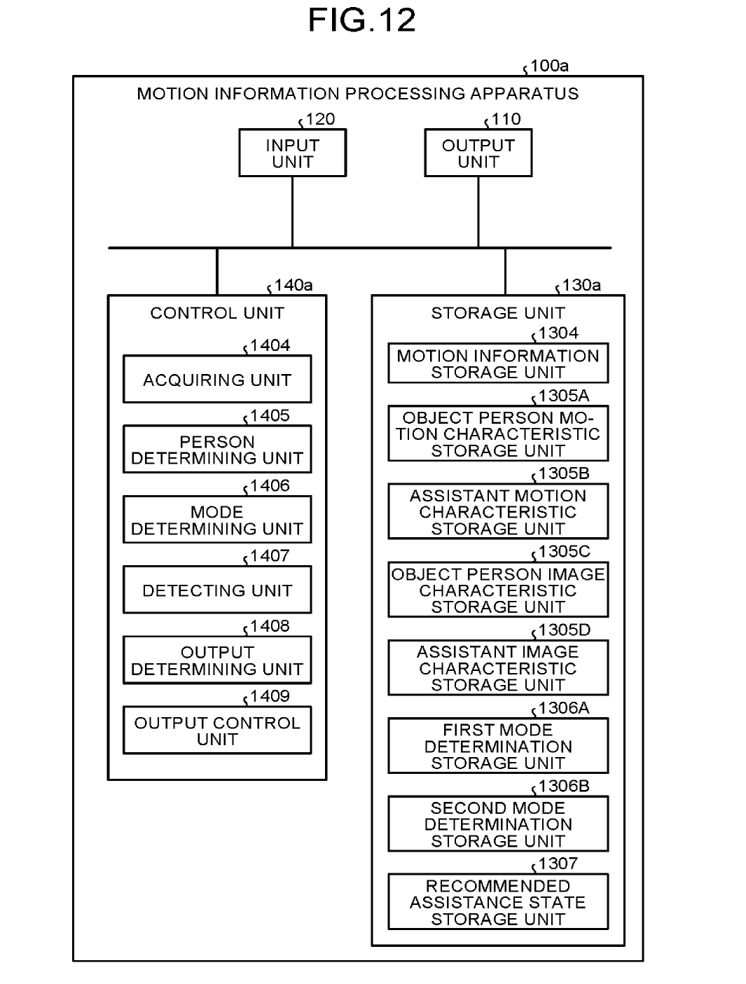

FIG. 12 is a block diagram of a detailed exemplary configuration of the motion information processing apparatus 100a according to the fifth embodiment. As illustrated in FIG. 12, a storage unit 130a in the motion information processing apparatus 100a includes a motion information storage unit 1304, an object person motion characteristic storage unit 1305A, an assistant motion characteristic storage unit 1305B, an object person image characteristic storage unit 1305C, an assistant image characteristic storage unit 1305D, a first mode determination storage unit 1306A, a second mode determination storage unit 13063, and a recommended assistance state storage unit 1307, for example.

The motion information storage unit 1304 stores therein various types of information acquired by the motion information acquiring unit 10. The motion information storage unit 1304, for example, stores therein information in which motion information, color image information, and audio recognition results are associated with one another about a motion of a person. The motion information is skeletal information of each frame generated by the motion information generating unit 14. The coordinates of the respective joints in the skeletal information are associated with the pixel positions in the color image information in advance. The capturing time information of the skeletal information is associated with the capturing time information of the color image information in advance. The motion information and the color image information are stored in the motion information storage unit 1304 every time being acquired by the motion information acquiring unit 10.

The motion information storage unit 1304, for example, stores therein motion information for each performed rehab, such as gait training and range of joint motion exercises. Single rehab may include motions of a plurality of persons. Specifically, in the case where an object person performs gait training with assistance given by an assistant as illustrated in FIG. 11, motions of the object person and the assistant are combined to perform single gait training. In this case, the motion information storage unit 1304 stores therein the pieces of skeletal information on the respective persons generated from distance image information of one frame in a manner associated with one another as a piece of motion information. In other words, the motion information indicates the motions of the persons simultaneously. The motion information storage unit 1304, for example, stores therein the motion information in association with capturing start time information indicating time when the capturing of the motion is started. While an explanation will be made of the case where the motion information indicates motions of a plurality of persons, the embodiment is not limited thereto. The motion information may indicate a motion of one person.

The object person motion characteristic storage unit 1305A stores therein object person motion characteristic information indicating characteristics of a motion of the object person. The object person motion characteristic storage unit 1305A, for example, stores therein information in which a motion identification (ID) is associated with object person motion characteristic information. The motion ID is identification information used to identify a motion and is created every time a designer of the motion information processing apparatus 100a defines a motion. The object person motion characteristic information is information indicating characteristics of a motion of the object person and is defined by the designer of the motion information processing apparatus 100a in advance.

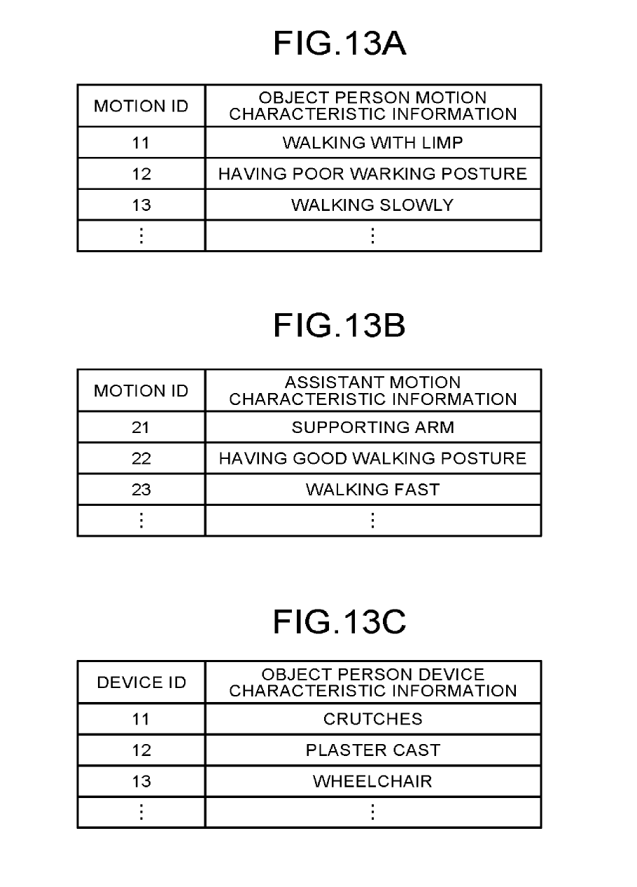

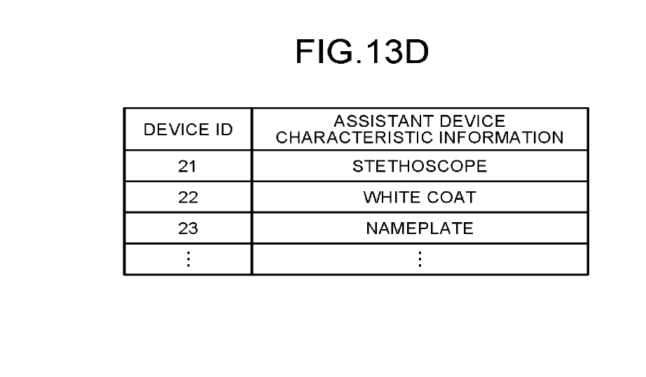

FIG. 13A is a diagram of an example of information stored in the object person motion characteristic storage unit 1305A. In the first record in FIG. 13A, a motion ID “11” is associated with object person motion characteristic information “walking with a limp”. In other words, the object person motion characteristic storage unit 1305A stores therein “walking with a limp”, which is one of the characteristics of the motion of the object person, as the motion of the motion ID “11”. The object person motion characteristic information “walking with a limp” is determined depending on whether the maximum change amount of the tarsus (the joint 2p or the joint 2t) in the y-coordinate during the motion is smaller than 1 cm, for example. In the second record in FIG. 13A, a motion ID “12” is associated with object person motion characteristic information “having a poor walking posture”. In other words, the object person motion characteristic storage unit 1305A stores therein “having a poor walking posture”, which is one of the characteristics of the motion of the object person, as the motion of the motion ID “12”. The object person motion characteristic information “having a poor walking posture” is determined depending on whether the average value of the angle formed by the spine (segment connecting the joint 2b and the joint 2c) and the vertical direction during the motion is equal to or larger than 3°, for example. In the third record in FIG. 13A, a motion ID “13” is associated with object person motion characteristic information “walking slowly”. In other words, the object person motion characteristic storage unit 1305A stores therein “walking slowly”, which is one of the characteristics of the motion of the object person, as the motion of the motion ID “13”. The object person motion characteristic information “walking slowly” is determined depending on whether the maximum value of the movement speed of the waist (joint 2c) during the motion is smaller than 1 (m/sec), for example. Similarly, the object person motion characteristic storage unit 1305A stores therein a motion ID and object person motion characteristic information in a manner associated with each other in other records. While the explanation has been made of the object person motion characteristic storage unit 1305A used in gait training as an example, the embodiment is not limited thereto. In a range of joint motion exercise, for example, the object person motion characteristic storage unit 1305A storing therein characteristics of the motion of the object person who performs the range of joint motion exercise may be used. Alternatively, the object person motion characteristic storage unit 1305A may store therein the characteristics of the motion of the object person who performs the gait training and the characteristics of the motion of the object person who performs the range of joint motion exercise with no distinction therebetween.

The assistant motion characteristic storage unit 1305B stores therein assistant motion characteristic information indicating characteristics of a motion of the assistant. The assistant motion characteristic storage unit 1305B, for example, stores therein information in which a motion ID is associated with assistant motion characteristic information. The assistant motion characteristic information is information indicating characteristics of a motion of the assistant and is defined by the designer of the motion information processing apparatus 100a in advance.

FIG. 13B is a diagram of an example of information stored in the assistant motion characteristic storage unit 1305B. In the first record in FIG. 13B, a motion ID “21” is associated with assistant motion characteristic information “supporting the arm”. In other words, the assistant motion characteristic storage unit 1305B stores therein “supporting the arm”, which is one of the characteristics of the motion of the assistant, as the motion of the motion ID “21”. The assistant motion characteristic information “supporting the arm” is determined depending on whether the hand (the joint 2h or the joint 21) of a first person is positioned within 5 cm from the arm (the segment connecting the joint 2e and the joint 2f or the segment connecting the joint 2i and the joint 2j) of a second person in a predetermined time during the motion, for example. In the second record in FIG. 13B, a motion ID “22” is associated with assistant motion characteristic information “having a good walking posture”. In other words, the assistant motion characteristic storage unit 1305B stores therein “having a poor walking posture”, which is one of the characteristics of the motion of the assistant, as the motion of the motion ID “22”. The assistant motion characteristic information “having a good walking posture” is determined depending on whether the average value of an angle formed by the spine (segment connecting the joint 2b and the joint 2c) and the vertical direction during the motion is smaller than 3°, for example. In the third record in FIG. 13B, a motion ID “23” is associated with assistant motion characteristic information “walking fast”. In other words, the assistant motion characteristic storage unit 1305B stores therein “walking fast”, which is one of the characteristics of the motion of the assistant, as the motion of the motion ID “23”. The assistant motion characteristic information “walking fast” is determined depending on whether the maximum value of the movement speed of the waist (joint 2c) during the motion is equal to or larger than 1 (m/sec), for example. Similarly, the assistant motion characteristic storage unit 1305B stores therein a motion ID and assistant motion characteristic information in a manner associated with each other in other records. While the explanation has been made of the assistant motion characteristic storage unit 1305B used in gait training as an example, the embodiment is not limited thereto. In a range of joint motion exercise, for example, the assistant motion characteristic storage unit 1305B storing therein characteristics of the motion of the assistant who performs the range of joint motion exercise may be used. Alternatively, the assistant motion characteristic storage unit 1305B may store therein the characteristics of the motion of the assistant who performs the gait training and the characteristics of the motion of the assistant who performs the range of joint motion exercise with no distinction therebetween.

The object person image characteristic storage unit 1305C stores therein object person image characteristic information indicating characteristics of an image of the object person. The object person image characteristic storage unit 1305C, for example, stores therein information in which a device ID is associated with object person device characteristics information. The device ID is identification information used to identify a device and is created every time the designer of the motion information processing apparatus 100a defines a device. The object person device characteristic information is information indicating characteristics of a device of the object person and image information on a device capable of being used in pattern matching, for example. The object person device characteristic information is defined by the designer of the motion information processing apparatus 100a in advance.

FIG. 13C is a diagram of an example of information stored in the object person image characteristic storage unit 1305C. In the first record in FIG. 13C, a device ID “11” is associated with object person device characteristic information “crutches”. In other words, the object person image characteristic storage unit 1305C stores therein image information “crutches”, which is one of the characteristics of the image of the object person, as the device of the device ID “11”. In the second record in FIG. 13C, a device ID “12” is associated with object person device characteristic information “plaster cast”. In other words, the object person image characteristic storage unit 1305C stores therein image information “plaster cast”, which is one of the characteristics of the image of the object person, as the device of the device ID “12”. In the third record in FIG. 13C, a device ID “13” is associated with object person device characteristic information “wheelchair”. In other words, the object person image characteristic storage unit 13050 stores therein image information “wheelchair”, which is one of the characteristics of the image of the object person, as the device of the device ID “13”. While the explanation has been made of the object person image characteristic storage unit 13050 used in gait training as an example, the embodiment is not limited thereto. In a range of joint motion exercise, for example, the object person image characteristic storage unit 1305C storing therein characteristics of the device of the object person who performs the range of joint motion exercise may be used. Alternatively, the object person image characteristic storage unit 1305C may store therein the characteristics of the device of the object person who performs the gait training and the characteristics of the device of the object person who performs the range of joint motion exercise with no distinction therebetween.

The assistant image characteristic storage unit 1305D stores therein assistant image characteristic information indicating characteristics of an image of the assistant. The assistant image characteristic storage unit 1305D, for example, stores therein information in which a device ID is associated with assistant device characteristics information. The assistant device characteristic information is information indicating characteristics of a device of the assistant and image information on a device capable of being used in pattern matching, for example. The assistant device characteristic information is defined by the designer of the motion information processing apparatus 100a in advance.

FIG. 13D is a diagram of an example of information stored in the assistant image characteristic storage unit 1305D. In the first record in FIG. 13D, a device ID “21” is associated with assistant device characteristic information “stethoscope”. In other words, the assistant image characteristic storage unit 1305D stores therein image information “stethoscope”, which is one of the characteristics of the image of the assistant, as the device of the device ID “21”. In the second record in FIG. 13D, a device ID “22” is associated with assistant device characteristic information “white coat”. In other words, the assistant image characteristic storage unit 1305D stores therein image information “white coat”, which is one of the characteristics of the image of the assistant, as the device of the device ID “22”. In the third record in FIG. 13D, a device ID “23” is associated with assistant device characteristic information “nameplate”. In other words, the assistant image characteristic storage unit 1305D stores therein image information “nameplate”, which is one of the characteristics of the image of the assistant, as the device of the device ID “23”.

The first mode determination storage unit 1306A and the second mode determination storage unit 1306B store therein information used to determine start and end of an assistance mode, which is a mode for supporting the assistant. The first mode determination storage unit 1306A and the second mode determination storage unit 1306B are referred to by a mode determining unit 1406, which will be described later, for example. The first mode determination storage unit 1306A and the second mode determination storage unit 1306B are registered in advance by a user of the motion information processing apparatus 100a, for example.

The first mode determination storage unit 1306A, for example, stores therein information in which an assistance mode determination motion is associated with an assistance mode determination result. The assistance mode determination motion is information indicating a motion used to determine the assistance mode. The assistance mode determination result is information indicating whether to start or terminate the assistance mode depending on the assistance mode determination motion. For example, “start” or “end” is stored as the assistance mode determination result.

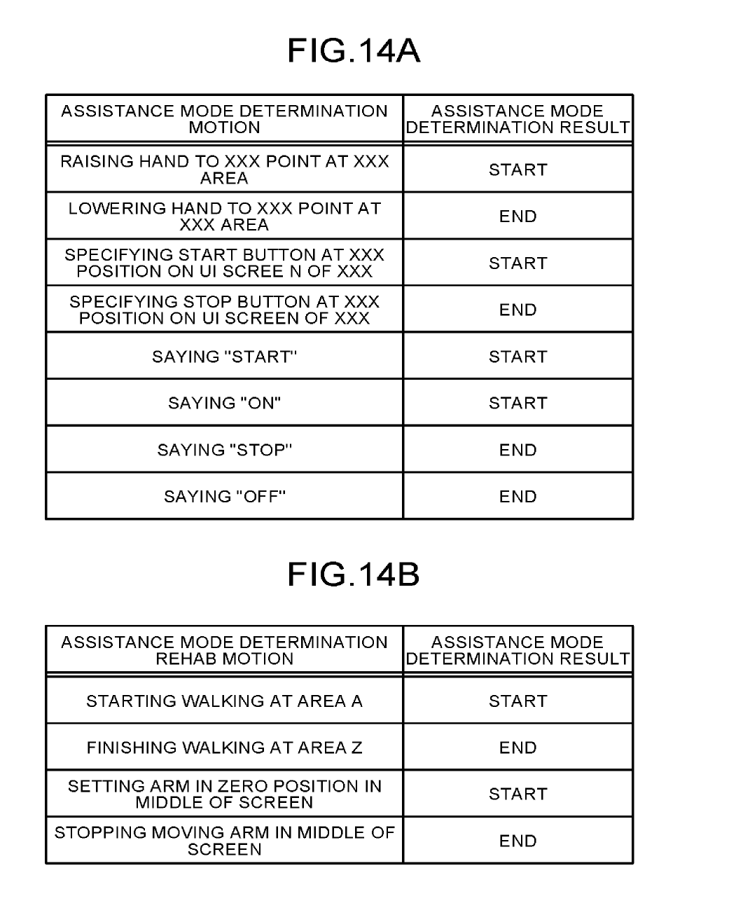

FIG. 14A is a diagram of an example of information stored in the first mode determination storage unit 1306A. In the first record in FIG. 14A, an assistance mode determination motion “raising the hand to a XXX point at a XXX area” is associated with an assistance mode determination result “start”. In other words, the first mode determination storage unit 1306A stores therein information that, if the motion of “raising the hand to a XXX point at a XXX area” is made, the assistance mode is started. In the second record in FIG. 14A, an assistance mode determination motion “lowering the hand to a XXX point at a XXX area” is associated with an assistance mode determination result “end”. In other words, the first mode determination storage unit 1306A stores therein information that, if the motion of “lowering the hand to a XXX point at a XXX area” is made, the assistance mode is terminated. Similarly, the first mode determination storage unit 1306A stores information in which an assistance mode determination motion is associated with an assistance mode determination result in other records.

The second mode determination storage unit 1306B, for example, stores therein information in which an assistance mode determination rehab motion is associated with an assistance mode determination result. The assistance mode determination rehab motion is information indicating a motion relating to rehab and used to determine the assistance mode.

FIG. 14B is a diagram of an example of information stored in the second mode determination storage unit 1306B. In the first record in FIG. 14B, an assistance mode determination rehab motion “starting walking at an area A” is associated with an assistance mode determination result “start”. In other words, the second mode determination storage unit 1306B stores therein information that, if the motion relating to rehab of “starting walking at an area A” is made, the assistance mode is started. In the second record in FIG. 14E, an assistance mode determination rehab motion “finishing walking at an area Z” is associated with an assistance mode determination result “end”. In other words, the second mode determination storage unit 1306B stores therein information that, if the motion relating to rehab of “finishing walking at an area Z” is made, the assistance mode is terminated. Similarly, the second mode determination storage unit 1306B stores information in which an assistance mode determination rehab motion is associated with an assistance mode determination result in other records. While the explanation has been made of the conditions in which persons are not identified as an example, persons may be identified if the persons are identifiable. In the case where an assistance mode determination rehab motion “the object person starts walking at an area A” is stored in the first record in FIG. 14B, for example, the second mode determination storage unit 1306B stores therein information that, if the motion relating to rehab of “the object person starts walking at an area A” is made, the assistance mode is started.

The recommended assistance state storage unit 1307 stores therein a recommended assistance state to support the assistant. The recommended assistance state storage unit 1307, for example, stores therein information in which an assistance stage, an assistance state, and a recommended assistance state are associated with one another. The assistance stage defines the degree of progress in a series of motions in rehab. The operator, for example, determines the assistance stage based on the state of assistance given by the assistant to the object person serving as a target of the rehab. The assistance state defines the state of assistance given by the assistant to the object person serving as a target of the rehab. The operator, for example, determines the assistance state based on the motion information on either one or both of the object person and the assistant. The recommended assistance state is information indicating the state of assistance recommended as the assistance given by the assistant to the object person and is registered for each assistance stage, for example. The recommended assistance state storage unit 1307, for example, stores therein the information for each type of rehab, such as gait training and range of joint motion exercises. The information stored in the recommended assistance state storage unit 1307 is registered in advance by the user of the motion information processing apparatus 100a based on suggestions of skilled assistants and object persons.

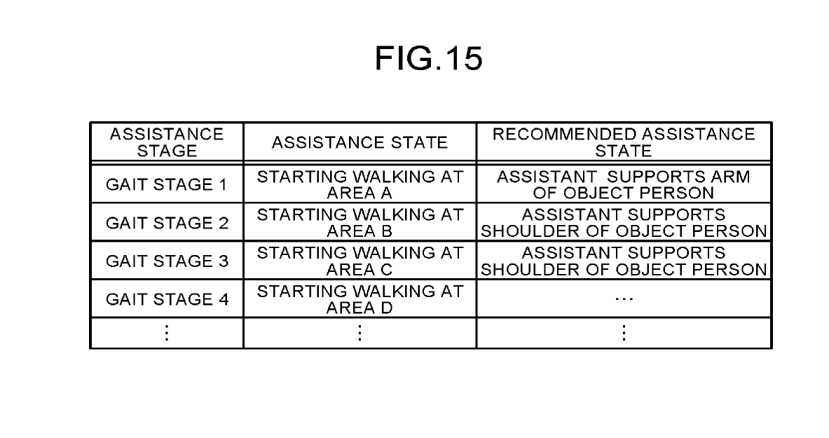

FIG. 15 is a diagram of an example of information stored in the recommended assistance state storage unit 1307. FIG. 15 illustrates a case where the recommended assistance state storage unit 1307 stores therein recommended assistance states relating to gait training, for example. In the first record in FIG. 15, an assistance stage “gait stage 1”, an assistance state “starting walking at an area A”, and a recommended assistance state “the assistant supports the arm of the object person” are associated with one another. In other words, the recommended assistance state storage unit 1307 stores therein information that the assistance stage “gait stage 1” in the gait training is a stage at which “starting walking at an area A” is performed and that the recommended motion made by the assistant for the object person is “the assistant supports the arm of the object person”. In the second record in FIG. 15, an assistance stage “gait stage 2”, an assistance state “starting walking at an area B”, and a recommended assistance state “the assistant supports the shoulder of the object person” are associated with one another. In other words, the recommended assistance state storage unit 1307 stores therein information that the assistance stage “gait stage 2” in the gait training is a stage at which “starting walking at an area B” is performed and that the recommended motion made by the assistant for the object person is “the assistant supports the shoulder of the object person”. The information stored in the recommended assistance state storage unit 1307 is not limited to the examples described above. If it is possible to identify the persons, for example, the persons may be identified to specify a motion of each person. Specifically, an assistance state “the object person and the assistant start walking at an area A” may be stored in the first record in FIG. 15.

While the motions, the states, and other elements of the persons are conceptually represented in the present embodiment, the motions and the states of the persons are specified based on the coordinates and the position relation of the joints in a plurality of consecutive frames.

Referring back to FIG. 12, a control unit 140 in the motion information processing apparatus 100a includes an acquiring unit 1404, a person determining unit 1405, the mode determining unit 1406, a detecting unit 1407, an output determining unit 1408, and an output control unit 1409.

The acquiring unit 1404 acquires motion information to be processed. If the acquiring unit 1404 receives an input to specify motion information to be processed from an input unit 120, for example, the acquiring unit 1404 acquires the specified motion information, color image information corresponding thereto, and an audio recognition result corresponding thereto from the motion information storage unit 1304.

If the acquiring unit 1404 receives specification of capturing start time information of motion information to be processed, for example, the acquiring unit 1404 acquires the motion information and color image information associated with the motion information from the motion information storage unit 1304. The motion information may include skeletal information on a plurality of persons or skeletal information on one person generated from distance image information of a signal frame.

The person determining unit 1405 determines whether the person corresponding to the motion information acquired by the acquiring unit 1404 is the object person. The person determining unit 1405 also determines whether the person corresponding to the motion information acquired by the acquiring unit 1404 is the assistant. If the motion information acquired by the acquiring unit 1404 includes skeletal information on a plurality of persons generated from distance image information of a signal frame, the person determining unit 1405 determines whether each piece of skeletal information on the persons corresponds to the object person or the assistant. The person determining unit 1405 outputs the determination result to the mode determining unit 1406. The processing of the person determining unit 1405 will be specifically described.

The following describes the processing for determining whether the person is the object person. The person determining unit 1405, for example, selects an unprocessed record out of the records in the object person motion characteristic storage unit 1305A and the object person image characteristic storage unit 1305C. The person determining unit 1405 then determines whether the acquired motion information and color image information correspond to the conditions of the selected record.

An explanation will be made of the case where the record of the motion ID “11” is selected from the object person motion characteristic storage unit 1305A. In this case, the person determining unit 1405 determines whether the motion information acquired by the acquiring unit 1404 corresponds to the object person motion characteristic information “walking with a limp” as illustrated in FIG. 13A. In other words, the person determining unit 1405 extracts the y-coordinate of the tarsus (the joint 2p or the joint 2t) from each frame included in the acquired motion information. The person determining unit 1405 then calculates the difference between the maximum value and the minimum value of the extracted y-coordinates as the maximum change amount. If the calculated maximum change amount is smaller than 1 cm, the person determining unit 1405 determines that the acquired motion information corresponds to the object person motion characteristic information, that is, that the person walks with a limp.

An explanation will be made of the case where the record of the motion ID “12” is selected from the object person motion characteristic storage unit 1305A. In this case, the person determining unit 1405 determines whether the motion information acquired by the acquiring unit 1404 corresponds to the object person motion characteristic information “having a poor walking posture” as illustrated in FIG. 13A. The person determining unit 1405, for example, extracts the coordinates of the joint 2b and the coordinates of the joint 2c of the person in each frame from the motion information acquired by the acquiring unit 1404. The person determining unit 1405 considers the segment connecting the joint 2b and the joint 2c, whose coordinates are extracted, as the spine of the person to derive the angle formed by the spine and the vertical direction in each frame. The person determining unit 1405 then calculates the average value of the angle in a plurality of frames during the gait training as the walking posture of the person. If the calculated walking posture is equal to or larger than 3°, the person determining unit 1405 determines that the acquired motion information corresponds to the object person motion characteristic information, that is, that the person has a poor walking posture.

An explanation will be made of the case where the record of the motion ID “13” is selected from the object person motion characteristic storage unit 1305A. In this case, the person determining unit 1405 determines whether the motion information acquired by the acquiring unit 1404 corresponds to the object person motion characteristic information “walking slowly” as illustrated in FIG. 13A. The person determining unit 1405, for example, derives the movement distance (m) of the coordinates of the joint 2c corresponding to the waist of the person every predetermined time (e.g., every 0.5 seconds). The person determining unit 1405 then calculates the movement speed (m/sec) of the person every predetermined time based on the movement distance per predetermined time. If the maximum movement speed of the calculated movement speed is smaller than 1 (m/sec), the person determining unit 1405 determines that the acquired motion information corresponds to the object person motion characteristic information, that is, that the person walks slowly.

An explanation will be made of the case where the record of the device ID “11” is selected from the object person image characteristic storage unit 1305C. In this case, the person determining unit 1405 performs pattern matching of the color image information acquired by the acquiring unit 1404 and the object person device characteristic information “crutches” as illustrated in FIG. 13C. If an image of crutches is extracted from the color image information by the pattern matching, the person determining unit 1405 determines whether the pixel positions of the extracted crutches overlap with the coordinates of the skeletal information included in the motion information to be processed. If the pixel positions of the crutches overlap with the coordinates of the skeletal information, the person determining unit 1405 determines that the acquired color image information corresponds to the object person device characteristic information, that is, that the person has the crutches. Similarly, the person determining unit 1405 determines whether the acquired color image information corresponds to object person device characteristic information in other records.

As described above, the person determining unit 1405 determines whether the acquired motion information and color image information correspond to the selected record. If it is determined that the motion information and the color image information correspond to the selected record, the person determining unit 1405 increments a retained object person characteristic number n by 1. The retained object person characteristic number n indicates the number of characteristics of the object person retained by the person corresponding to the motion information to be processed. Similarly, the person determining unit 1405 determines whether the acquired motion information and color image information correspond to other unprocessed records. If the retained object person characteristic number n reaches 5, the person determining unit 1405 determines that the person corresponding to the motion information to be processed is the object person. By contrast, if determination is made for all the records in the object person motion characteristic storage unit 1305A and the object person image characteristic storage unit 1305C but the retained object person characteristic number n does not reach 5, the person determining unit 1405 determines that the person corresponding to the motion information to be processed is not the object person. While the threshold of the retained object person characteristic number n used to determine whether the person is the object person is set to “5” in this example, the embodiment is not limited thereto. The threshold may be set to any desired value by the operator. While the explanation has been made of the case where, if the motion information and the color image information correspond to each record, the retained object person characteristic number n is incremented by 1, the embodiment is not limited thereto. Each record may be weighted, for example.

The following describes the processing for determining whether the person is the assistant. The person determining unit 1405, for example, selects an unprocessed record out of the records in the assistant motion characteristic storage unit 1305B and the assistant image characteristic storage unit 1305D. The person determining unit 1405 then determines whether the acquired motion information and color image information correspond to the selected record.

An explanation will be made of the case where the record of the motion ID “21” is selected from the assistant motion characteristic storage unit 1305B. In this case, the person determining unit 1405 determines whether the motion information acquired by the acquiring unit 1404 corresponds to the assistant motion characteristic information “supporting the arm” as illustrated in FIG. 13B. In other words, the person determining unit 1405 acquires the coordinates of the hand (the joint 2h or the joint 21) from each frame included in the acquired motion information. If the arm (the segment connecting the joint 2e and the joint 2f or the segment connecting the joint 2i and the joint 2j) of the second person is positioned within 5 cm from the hand, whose coordinates are acquired, in a predetermined time during the gait training, the person determining unit 1405 determines that the acquired motion information corresponds to the assistant motion characteristic information, that is, that the person supports the arm.

An explanation will be made of the case where the record of the motion ID “22” is selected from the assistant motion characteristic storage unit 1305B. In this case, the person determining unit. 1405 determines whether the motion information acquired by the acquiring unit 1404 corresponds to the assistant motion characteristic information “having a good walking posture” as illustrated in FIG. 13B. The person determining unit 1405, for example, calculates the walking posture of the person in the same manner as described above. If the calculated walking posture is smaller than 3°, the person determining unit 1405 determines that the acquired motion information corresponds to the assistant motion characteristic information, that is, that the person has a good walking posture.

An explanation will be made of the case where the record of the motion ID “23” is selected from the assistant motion characteristic storage unit 1305B. In this case, the person determining unit 1405 determines whether the motion information acquired by the acquiring unit 1404 corresponds to the assistant motion characteristic information “walking fast” as illustrated in FIG. 13B. The person determining unit 1405, for example, calculates the movement speed (m/sec) of the person every predetermined time (e.g., 0.5 seconds) in the same manner as described above. If the maximum movement speed of the calculated movement speed is equal to or larger than 1 (m/sec), the person determining unit 1405 determines that the acquired motion information corresponds to the assistant motion characteristic information, that is, that the person walks fast.

An explanation will be made of the case where the record of the device ID “21” is selected from the assistant image characteristic storage unit 1305D. In this case, the person determining unit 1405 performs pattern matching of the color image information acquired by the acquiring unit 1404 and the assistant device characteristic information “stethoscope” as illustrated in FIG. 13D. If an image of a stethoscope is extracted from the color image information by the pattern matching, the person determining unit 1405 determines whether the pixel positions of the extracted stethoscope overlap with the coordinates of the skeletal information included in the motion information to be processed. If the pixel positions of the stethoscope overlap with the coordinates of the skeletal information, the person determining unit 1405 determines that the acquired color image information corresponds to the assistant device characteristic information, that is, that the person has the stethoscope. Similarly, the person determining unit 1405 determines whether the acquired color image information corresponds to assistant device characteristic information in other records.

As described above, the person determining unit 1405 determines whether the acquired motion information and color image information correspond to the selected record. If it is determined that the motion information and the color image information correspond to the selected record, the person determining unit 1405 increments a retained assistant characteristic number m by 1. The retained assistant characteristic number m indicates the number of characteristics of the assistant retained by the person corresponding to the motion information to be processed. Similarly, the person determining unit 1405 determines whether the acquired motion information and color image information correspond to other unprocessed records. If the retained assistant characteristic number m reaches 5, the person determining unit 1405 determines that the person corresponding to the motion information to be processed is the assistant. By contrast, if determination is made for all the records in the assistant motion characteristic storage unit 1305B and the assistant image characteristic storage unit 1305D but the retained assistant characteristic number m does not reach 5, the person determining unit 1405 determines that the person corresponding to the motion information to be processed is not the assistant. While the threshold of the retained assistant characteristic number n used to determine whether the person is the assistant is set to “5” in this example, the embodiment is not limited thereto. The threshold may be set to any desired value by the operator. While the explanation has been made of the case where, if the motion information and the color image information correspond to each record, the retained assistant characteristic number m is incremented by 1, the embodiment is not limited thereto. Each record may be weighted, for example.

The processing of the person determining unit 1405 is not limited to the processing described above. If a plurality of persons are captured in the color image information acquired by the acquiring unit 1404, for example, the person determining unit 1405 may make the determination based on the positions of the persons in the color image information. Alternatively, by providing an identification marker used to identify a person to either one or both of the object person and the assistant, the person determining unit 1405 may make the determination using the identification marker included in the color image information or the distance image information, for example. Examples of the identification marker include a marker identifiable from the color image information by pattern matching and a marker whose position in a space is identifiable by a magnetic sensor.

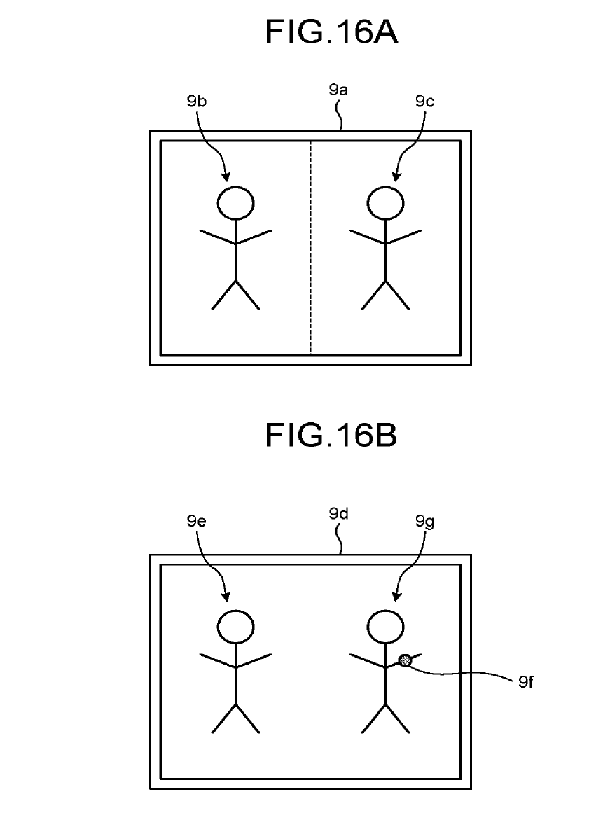

FIG. 16A is a view for explaining determination processing performed by the person determining unit 1405 based on positions of persons. FIG. 16A illustrates a screen 9a of the motion information processing apparatus 100a displaying an image in which a person 9b and a person 9c are performing rehab, for example. In this case, the person determining unit 1405 determines that the person 9b on the left captured in the color image is the object person and that the person 9c on the right is the assistant, for example. This determination method is effectively used particularly when the space in which the rehab is performed and the position of the motion information acquiring unit 10 are determined in advance and when the direction in which the assistant gives assistance to the object person is determined, for example. Specifically, in the case where the object person performs gait training while holding on to a hand rail arranged on a wall with the right hand, the assistant gives assistance to the object person from the left.

FIG. 16B is a view for explaining determination processing performed by the person determining unit 1405 using an identification marker. FIG. 16B illustrates a screen 9d of the motion information processing apparatus 100a displaying an image in which a person 9e and a person 9g wearing an identification marker 9f are performing rehab, for example. In this case, the person determining unit 1405 determines that the person 9g wearing the identification marker 9f is the assistant and that the person 9e not wearing the identification marker 9f is the object person, for example. The embodiment is not limited to this example, and the identification marker may be attached to the object person or both the object person and the assistant, for example. This determination method is effectively used particularly when a person who works as an assistant and an object person who frequently performs rehab are present in a facility where the rehab is performed, for example.

As described above, the person determining unit 1405 determines whether the person corresponding to the motion information to be processed is the object person or the assistant and outputs the determination result to the mode determining unit 1406. If the person determining unit 1405 determines that the person corresponding to the motion information to be processed is neither the object person nor the assistant, the person determining unit 1405 outputs a determination result indicating that the person cannot be determined to the detecting unit 1407. If the motion information to be processed includes skeletal information on a plurality of persons, the person determining unit 1405 determines whether each of the persons is the object person or the assistant.

The mode determining unit 1406 determines start and end of the assistance mode, which is a mode for supporting the assistant. The mode determining unit 1406, for example, determines start and end of the assistance mode depending on whether the motion information acquired by the acquiring unit 1404 corresponds to the conditions included in the assistance mode determination motions in the first mode determination storage unit 1306A or the assistance mode determination rehab motions in the second mode determination storage unit 1306B.

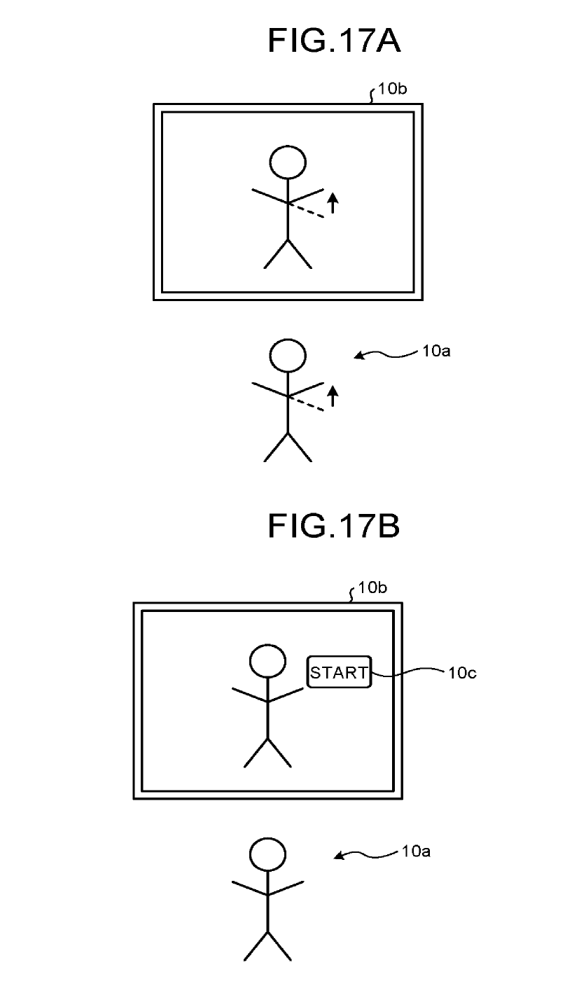

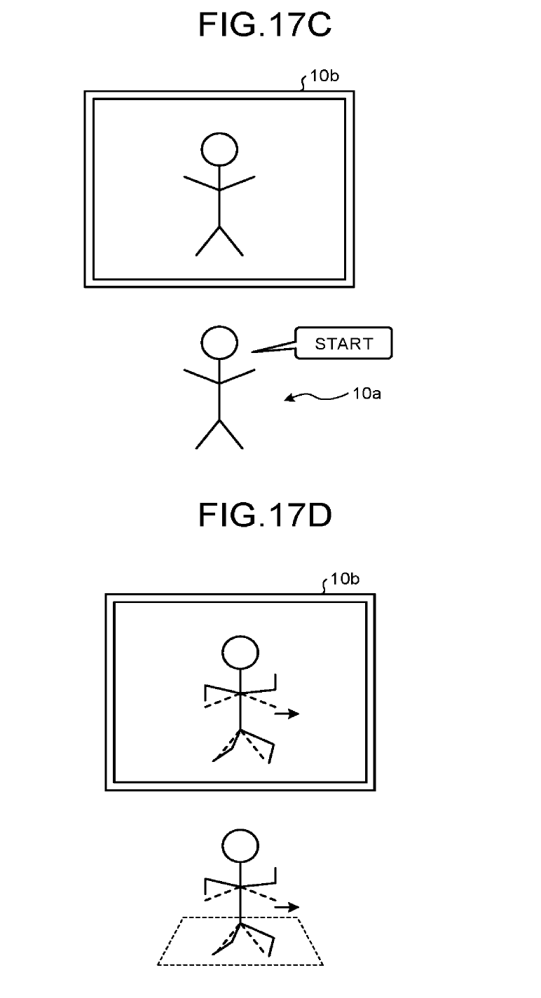

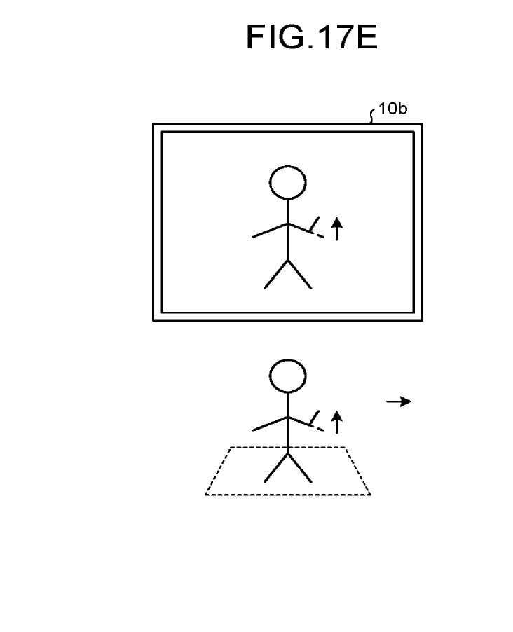

FIG. 17A to FIG. 17E are views for explaining the processing of the mode determining unit 1406. FIG. 17A to FIG. 17C each illustrate a case where the mode determining unit 1406 determines start and end of the assistance mode using the first mode determination storage unit 1306A. FIG. 17D and FIG. 17E each illustrate a case where the mode determining unit 1406 determines start and end of the assistance mode using the second mode determination storage unit 1306B.

FIG. 17A illustrates an example in which detection of a predetermined motion starts the assistance mode. The first mode determination storage unit 1306A stores therein information in which an assistance mode determination motion “raising the right hand in the middle of the screen” is associated with the assistance mode determination result “start”. In this case, if the mode determining unit 1406 detects that a person 10a raises the right hand at a position corresponding to the middle of a screen 10b (e.g., the y-coordinate of the joint 2h of the right hand is positioned higher than the y-coordinate of the joint 2e of the right shoulder) in the space where rehab is performed, for example, the mode determining unit 1406 determines to start the assistance mode.

FIG. 17B illustrates an example in which use of an operation button on the screen starts the assistance mode. The first mode determination storage unit 1306A stores therein information in which an assistance mode determination motion “a start button in the screen is specified” is associated with the assistance mode determination result “start”. In this case, if the mode determining unit 1406 detects that the person 10a extends the right hand to a position corresponding to a start button 10c in the screen 10b (the coordinates of the joint 2h overlap with the position of the start button 10c) in the space where rehab is performed, for example, the mode determining unit 1406 determines to start the assistance mode.

FIG. 17C illustrates an example in which use of audio starts the assistance mode. The first mode determination storage unit 1306A stores therein information in which an assistance mode determination motion “saying “start”” is associated with the assistance mode determination result “start”. In this case, if the mode determining unit 1406 detects that the person 10a says the word “start” from an audio recognition result recognized in the space where rehab is performed, for example, the mode determining unit 1406 determines to start the assistance mode.

FIG. 17D illustrates an example in which detection of a predetermined rehab motion starts the assistance mode. The second mode determination storage unit 1306B stores therein information in which an assistance mode determination rehab motion “starting walking at an area A” is associated with the assistance mode determination result “start”. In this case, if the mode determining unit 1406 detects that the person 10a starts gait training at a position corresponding to the area A in the screen 10b in the space where the rehab is performed, for example, the mode determining unit 1406 determines to start the assistance mode.

FIG. 17E illustrates an example in which detection of a predetermined rehab motion starts the assistance mode. The first mode determination storage unit 1306B stores therein information in which an assistance mode determination rehab motion “setting the arm in a zero position in the middle of the screen” is associated with the assistance mode determination result “start”. In this case, if the mode determining unit 1406 detects that the person 10a sets the right arm in the zero position to perform a range of joint motion exercise of the right arm at a position corresponding to the middle of the screen 10b in the space where the rehab is performed, for example, the mode determining unit 1406 determines to start the assistance mode. The zero position represents an initial state of a joint serving as a target of a range of joint motion exercise in which the joint is bent and stretched, for example. In a range of joint motion exercise of the right elbow, the zero position is a state where the right elbow is stretched straight (the angle formed by the joint 2f is 180°), for example.

As described above, the mode determining unit 1406 refers to the first mode determination storage unit 1306A or the second mode determination storage unit 1306B, thereby determining start and end of the assistance mode. The processing of the mode determining unit 1406 is not limited to the processing described above. The mode determining unit 1406 may make the determination using movement of a viewpoint, a direction of the face, acceleration of the hand, a body motion, frequency of conversations, and time, for example.

The detecting unit 1407 detects an assistance state indicating the state of assistance given by the assistant to the object person serving as a target of rehabilitation based on the motion information acquired by the acquiring unit 1404. The detecting unit 1407, for example, detects an assistance state including at least one of a positional relation between the object person and the assistant, movement states of the object person and the assistant, and an instruction action performed by the assistant to the object person. The detecting unit 1407, for example, detects a positional relation between the object person and the assistant, movement states of the object person and the assistant, an assistance action performed by the assistant for the object person, and explicit actions of an executor and the assistant as an assistance state. The detecting unit 1407 then detects one of or a combination of the positional relation, the movement states, the assistance action, and the explicit actions as an assistance state created by the assistant for the object person. The processing of the detecting unit 1407 is performed on the motion information in which at least one object person and one assistant are identified by the person determining unit 1405. In the following description, an assumption is made that the object person and the assistant are identified.

The following describes the positional relation between the object person and the assistant detected by the detecting unit 1407. The detecting unit 1407, for example, extracts the position of the waist of the object person (coordinates of the joint 2c) and the position of the waist of the assistant (coordinates of the joint 2c) in each frame from the motion information acquired by the acquiring unit 1404. The detecting unit 1407 calculates a relative distance between the position of the waist of the object person (coordinates of the joint 2c) and the position of the waist of the assistant (coordinates of the joint 2c). The detecting unit 1407 detects the position of the waist of the object person, the position of the waist of the assistant, and the relative distance therebetween as the positional relation between the object person and the assistant, for example.

The following describes the movement states of the object person and the assistant detected by the detecting unit 1407. The detecting unit 1407, for example, derives movement distances (m) of the positions of the waists of the object person and the assistant (coordinates of the joint 2c) every predetermined time (e.g., 0.5 seconds). The detecting unit 1407 calculates the movement speed and the acceleration of the object person and the assistant based on the movement distances per predetermined time. The detecting unit 1407 detects the calculated movement speed and acceleration of the object person and the assistant as the movement states of the object person and the assistant.

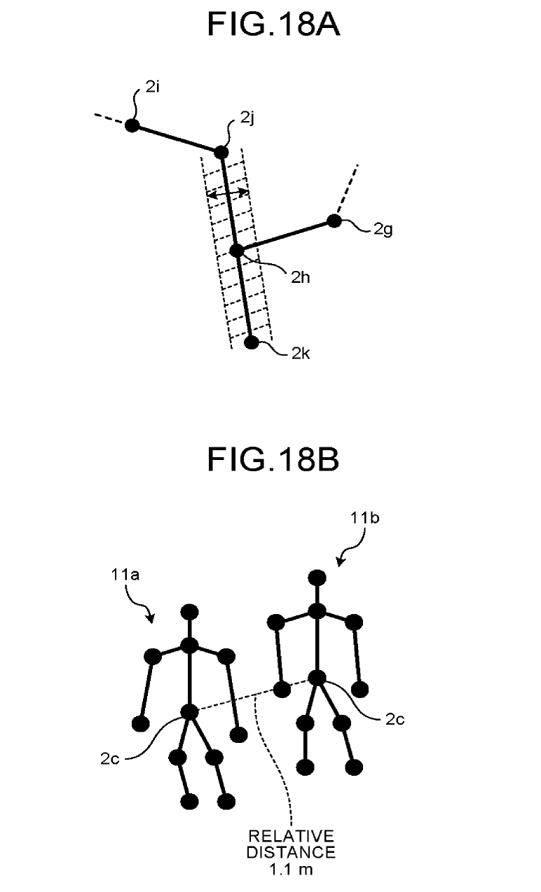

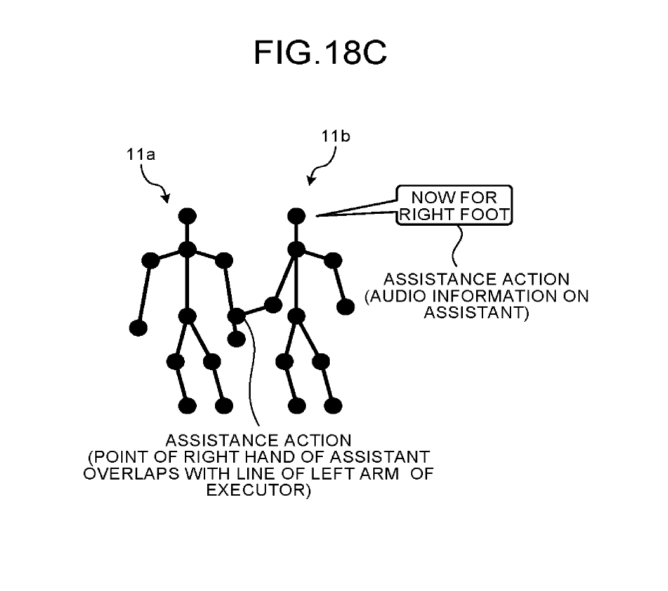

The following describes the assistance action performed by the assistant for the object person, which assistance action is detected by the detecting unit 1407. The detecting unit 1407, for example, detects an assistance action from the motion information acquired by the acquiring unit 1404. The assistance action includes a positional relation of a contact position of the object person and the assistant and audio of the assistant. FIG. 18A is a view for explaining the processing of the detecting unit 1407. FIG. 18A illustrates the joints 2i, 2j, and 2k of the object person and the joints 2h and 2g of the assistant. As illustrated in FIG. 18A, the detecting unit 1407 detects that the joint 2h of the right hand of the assistant is present within a predetermined distance from the left arm of the object person (segment connecting the joint 2i and the joint 2j) in each frame. As a result, the detecting unit 1407 detects a state in which “the right hand of the assistant holds the left arm of the object person”. If audio output from the vicinity of the head of the assistant is recognized at time corresponding to a frame, the detecting unit 1407 detects the audio as audio of the assistant. The detecting unit 1407 detects the state in which “the right hand of the assistant holds the left arm of the object person” and the audio of the assistant as the assistance action performed by the assistant for the object person.

The following describes the explicit actions of the object person and the assistant detected by the detecting unit 1407. The explicit actions define an explicit motion unique to the object person and an explicit motion unique to the assistant. The motion of the assistant to assist (support) the object person out of the actions of the assistant is included in the assistance action described above, and the explicit action of the assistant includes actions other than the assistance action. The detecting unit 1407, for example, detects an explicit motion unique to the object person and an explicit motion unique to the assistant from the motion information acquired by the acquiring unit 1404. Examples of the explicit motion unique to the object person include walking with a limp. Examples of the explicit motion unique to the assistant include taking notes at predetermined time intervals. Such explicit motions unique to the object person and explicit motions unique to the assistant are registered by the user in advance. The detecting unit 1407 detects the explicit motion unique to the object person and the detected explicit motion unique to the assistant as the explicit actions of the executor and the assistant.

The processing of the detecting unit 1407 for detecting an assistance state will be specifically described. FIG. 18B and FIG. 18C are views for explaining the processing of the detecting unit 1407. In the example illustrated in FIG. 18B, the detecting unit 1407 detects the position of a object person 11a, the position of an assistant 11b, and a relative distance (1.1 m) between the object person 11a and the assistant 11b as the positional relation between the object person 11a and the assistant 11b. The detecting unit 1407 also detects that the object person 11a and the assistant 11b are moving at predetermined speed from the rear side to the front side as the movement state of the object person 11a and the assistant 11b. Thus, the detecting unit 1407 detects an assistance state in which “the assistant 11b is walking beside the object person 11a”. As illustrated in FIG. 18B, the detecting unit 1407 does not necessarily use all of the positional relation between the object person and the assistant, the movement states of the object person and the assistant, the assistance action performed by the assistant for the object person, and the explicit actions of the executor and the assistant.

In the example illustrated in FIG. 18C, the detecting unit 1407 detects the position of the object person 11a, the position of the assistant 11b, and a relative distance between the object person 11a and the assistant 11b as the positional relation between the object person 11a and the assistant 11b. The detecting unit 1407 also detects a state in which “the right hand of the assistant 11b holds the left arm of the object person 11a” and audio “now for the right foot” made by the assistant 11b as an assistance action performed by the assistant 11b for the object person 11a. Thus, the detecting unit 1407 detects an assistance state in which “the assistant 11b is saying, “Now for the right foot.” to the object person 11a while holding the left arm of the object person 11a with the right hand”.

As described above, the detecting unit 1407 uses at least one of or a combination of the positional relation, the movement states, the assistance action, and the explicit actions, thereby detecting an assistance state created by the assistant for the object person. The detecting unit 1407 outputs the detected assistance state to the output determining unit 1408.

The output determining unit 1408 determines whether the assistance state detected by the detecting unit 1407 satisfies a recommended assistance state. The output determining unit 1408, for example, receives the assistance state detected by the detecting unit 1407. The output determining unit 1408 refers to the recommended assistance state storage unit 1307, thereby identifying an assistance stage corresponding to the received assistance state. The output determining unit 1408 compares the received assistance state with the recommended assistance state corresponding to the identified assistance stage, thereby determining whether the assistance state satisfies the recommended assistance state.

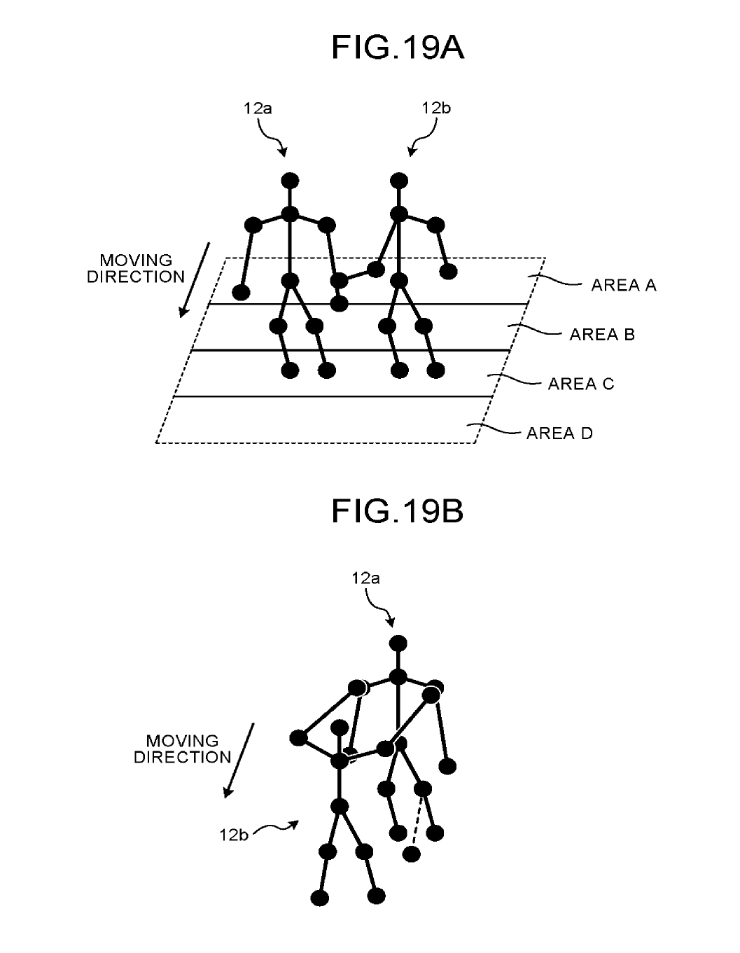

FIG. 19A and FIG. 19B are views for explaining the processing of the output determining unit 1408. FIG. 19A and the FIG. 19B each illustrate a state of rehab displayed on the screen of the motion information processing apparatus 100a.

In the example illustrated in FIG. 19A, the recommended assistance state storage unit 1307 stores therein information in which an assistance stage “gait stage 3”, an assistance state “starting walking at area C”, and a recommended assistance state in which “the assistant supports the shoulder of the object person” are associated with one another. As illustrated in FIG. 19A, the output determining unit 1408 receives an assistance state in which “gait training is being performed while an assistant 12b is supporting the left arm of a object person 12a with the right hand at an area C”. Because the received assistance state in which “gait training is being performed while the assistant 12b is supporting the left arm of the object person 12a with the right hand at an area C” satisfies the assistance state “starting walking at area C”, the output determining unit 1408 identifies the assistance stage corresponding to the received assistance state as the assistance stage “gait stage 3”. The output determining unit 1408 compares the received assistance state in which “gait training is being performed while the assistant 12b is supporting the left arm of the object person 12a with the right hand at an area C” with the recommended assistance state of the “gait stage 3,” that is, “the assistant supports the shoulder of the object person”. Because the assistant 12b is supporting the left arm of the object person 12a, the output determining unit 1408 determines that the received assistance state does not satisfy the recommended assistance state.

In the example illustrated in FIG. 19B, the recommended assistance state storage unit 1307 stores therein information in which an assistance stage “gait stage 2”, an assistance state in which “the assistant is supporting the shoulders of the object person from the front”, and a recommended assistance state in which “the assistant moves before the object person does” are associated with one another. As illustrated in FIG. 19B, the output determining unit 1408 receives an assistance state in which “the assistant 12b stops to support the shoulders of the object person 12a from the front, and the object person 12a is about to walk”. Because the received assistance state in which “the assistant 12b stops to support the shoulders of the object person 12a from the front, and the object person 12a is about to walk” satisfies the assistance state in which “the assistant is supporting the shoulders of the object person from the front”, the output determining unit 1408 identifies the assistance stage corresponding to the received assistance state as the assistance stage “gait stage 2”. The output determining unit 1408 compares the received assistance state in which “the assistant 12b stops to support the shoulders of the object person 12a from the front, and the object person 12a is about to walk” with the recommended assistance state of “gait stage 2”, that is, “the assistant moves before the object person does”. Because the object person 12a is about to walk even though the assistant 12b stops walking, the output determining unit 1408 determines that the received assistance state does not satisfy the recommended assistance state. The state “being about to walk” means an increase in the acceleration of the knee joint, for example, and is detected by the detecting unit 1407.

As described above, the output determining unit 1408 determines whether the assistance state detected by the detecting unit 1407 satisfies the recommended assistance state. The output determining unit 1408 outputs the determination result to the output control unit 1409.

The output control unit 1409 outputs assistance support information used to support the assistant based on the assistance state detected by the detecting unit 1407. The output control unit 1409, for example, outputs assistance support information based on the determination result of the output determining unit 1408.

The output control unit 1409, for example, receives a determination result indicating that the assistance state detected by the detecting unit 1407 does not satisfy the recommended assistance state from the output determining unit 1408. In this case, the output control unit 1409 outputs information indicating the recommended assistance state to an output unit 110 as the assistance support information, for example. Specifically, the output control unit 1409 causes a monitor and a glasses-like display worn by the assistant to display an image indicating the recommended assistance state in which “the assistant supports the shoulder of the object person”, for example. The output control unit 1409 causes a speaker or a headset worn by the assistant to output audio indicating the recommended assistance state in which “the assistant supports the shoulder of the object person”. The assistance support information output by the output control unit 1409 may be a warning sound besides information indicating the recommended assistance state.

The output control unit 1409, for example, may calculate the difference between the assistance state and the recommended assistance state from the comparison result between the assistance state and the recommended assistance state, thereby outputting the calculated difference as the assistance support information. Specifically, the output control unit 1409 acquires the assistance state and the recommended assistance state used for the determination from the output determining unit 1408. The output control unit 1409 then derives information indicating what kind of motion the assistant has to make to achieve the recommended assistance state. In the example illustrated in FIG. 19A, the output control unit 1409 subtracts the coordinates (xh2, yh2, zh2) of the right hand (joint 2h) of the assistant in the assistance state from the coordinates (xh1, yh1, zh1) of the right hand (joint 2h) of the assistant in the recommended assistance state. As a result, the output control unit 1409 derives the fact that moving the right hand (joint 2h) of the assistant by xh1-xh2 in the x-axis direction, yh1-yh2 in the y-axis direction, and zh1-zh2 in the z-axis direction achieves the recommended assistance state. The output control unit 1409 displays the calculated difference on the monitor and the glasses-like display as an image or outputs the calculated difference as audio. The output control unit 1409, for example, displays the calculation result as an arrow directed from the current position of the right hand to the position of the right hand in the recommended assistance state. Alternatively, the output control unit 1409 may express the calculation result in a direction viewed from the assistant, thereby outputting audio of “Move the right hand by 5 cm to the right, 20 cm upward, and 3 cm to the back.”, for example.

The output control unit 1409, for example, receives a determination result indicating that the assistance state detected by the detecting unit 1407 satisfies the recommended assistance state from the output determining unit 1408. In this case, the output control unit 1409 outputs information indicating that the recommended assistance state is satisfied as the assistance support information to the output unit 110, for example. Specifically, the output control unit 1409 causes the monitor and the glasses-like display worn by the assistant to display characters “Good” as the information indicating that the recommended assistance state is satisfied, for example. The output control unit 1409 causes the speaker or the headset worn by the assistant to output audio “Good” for indicating that the recommended assistance state is satisfied. If the assistance state detected by the detecting unit 1407 satisfies the recommended assistance state, the output control unit 1409 does not necessarily output the assistance support information.

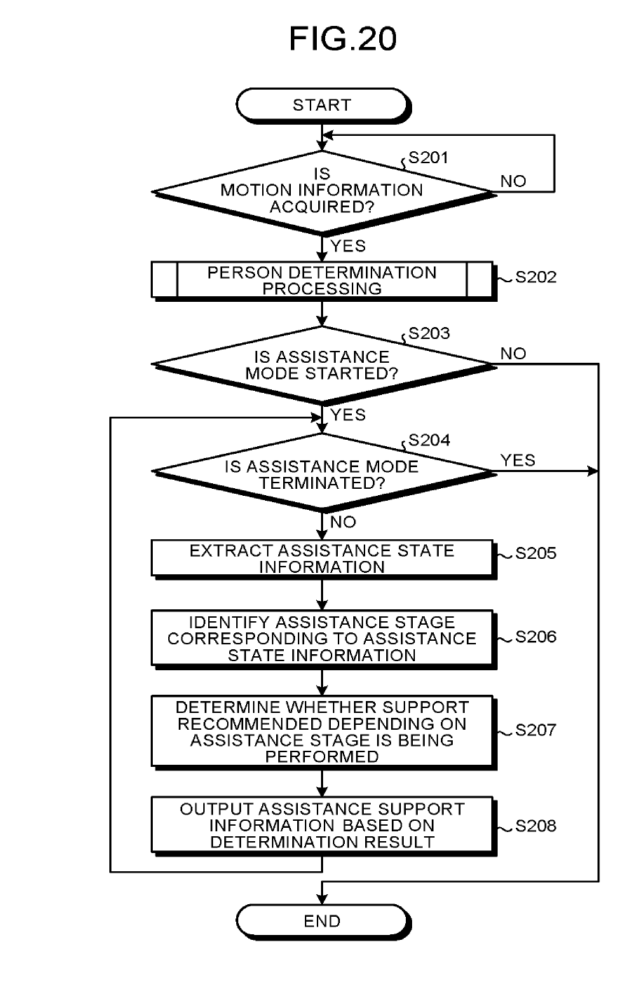

The following describes a processing procedure of the motion information processing apparatus 100a according to the fifth embodiment with reference to FIG. 20. FIG. 20 is a flowchart for explaining an example of a processing procedure of the motion information processing apparatus 100a according to the fifth embodiment.

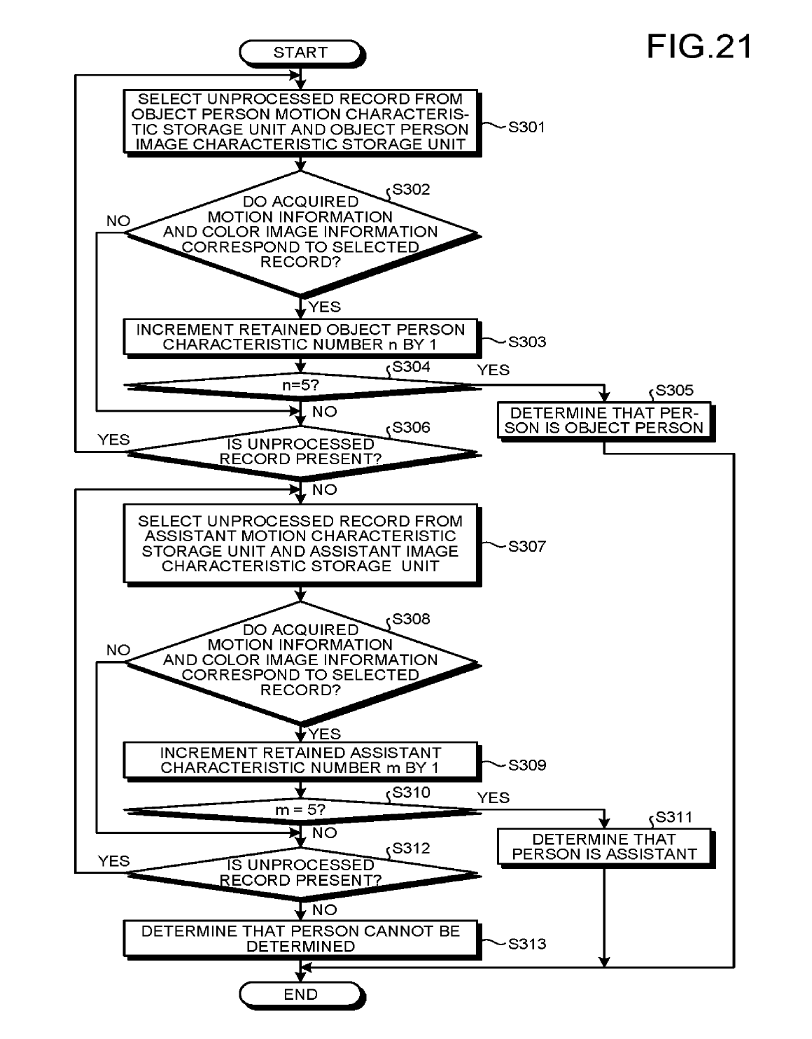

As illustrated in FIG. 20, if the acquiring unit 1404 acquires motion information to be processed (Yes at Step S201), the person determining unit 1405 performs person determination processing (Step S202). The person determination processing will be described later with reference to FIG. 21. Until the acquiring unit 1404 acquires motion information to be processed (No at Step S201), the motion information processing apparatus 100a remains in a standby state.

Subsequently, the mode determining unit 1406 determines whether the assistance mode is started (Step S203). If the assistance mode is started (Yes at Step S203), the mode determining unit 1406 determines whether the assistance mode is terminated (Step S204). If the assistance mode is not started (No at Step S203) or if the assistance mode is terminated (Yes at Step S204), the motion information processing apparatus 100a operates in a mode other than the assistance mode, such as a mode for supporting the object person by detecting a motion of the object person. Because any known processing may be used for the processing procedure of this motion, the explanation thereof will be omitted.

If the assistance mode is not terminated (No at Step S204), the detecting unit 1407 detects an assistance state in each frame based on the motion information acquired by the acquiring unit 1404 (Step S205). The detecting unit 1407, for example, uses at least one of or a combination of a positional relation, movement states, an assistance action, and explicit actions, thereby detecting an assistance state created by the assistant for the object person.

Subsequently, the output determining unit 1408 receives the assistance state detected by the detecting unit 1407, thereby identifying an assistance stage corresponding to the received assistance state (Step S206). The output determining unit 1408 determines whether support recommended depending on the assistance stage is being performed (Step S207). The output determining unit 1408, for example, compares the received assistance state to a recommended assistance state corresponding to the identified assistance stage, thereby determining whether the assistance state satisfies the recommended assistance state.

The output control unit 1409 outputs assistance support information based on the determination result of the output determining unit 1408 (Step S208). If the assistance state detected by the detecting unit 1407 satisfies the recommended assistance state, the output control unit 1409 does not necessarily output the assistance support information.

The processing is not necessarily performed in the order described above. The processing for performing the person determination processing at Step S202 may be performed after the processing for determining whether the assistance mode is started at Step S203, for example.

The person determination processing at Step S202 will now be described with reference to FIG. 21. FIG. 21 is a flowchart for explaining an example of a processing procedure of the person determination processing according to the fifth embodiment.

The person determining unit 1405 selects an unprocessed record from the object person motion characteristic storage unit 1305A and the object person image characteristic storage unit 1305C (Step S301). The person determining unit 1405 then determines whether the acquired motion information and color image information correspond to the selected record (Step S302). If the motion information and the color image information correspond to the selected record (Yes at Step S302), the person determining unit 1405 increments the retained object person characteristic number n by 1 (Step S303). The person determining unit 1405 then determines whether the retained object person characteristic number n reaches 5 (Step S304). If the retained object person characteristic number n reaches 5 (Yes at Step S304), the person determining unit 1405 determines that the person corresponding to the motion information acquired by the acquiring unit 1404 is the object person (Step S305).

By contrast, if the retained object person characteristic number n does not reach 5 (No at Step S304), the person determining unit 1405 determines whether an unprocessed record is present in the object person motion characteristic storage unit and the object person image characteristic storage unit (Step S306). If an unprocessed record is present (Yes at Step S306), the person determining unit 1405 repeats the processing from Step S301.

By contrast, if no unprocessed record is present (No at Step S306), the person determining unit 1405 selects an unprocessed record from the assistant motion characteristic storage unit 1305B and the assistant image characteristic storage unit 1305D (Step S307). The person determining unit 1405 then determines whether the acquired motion information and color image information correspond to the selected record (Step S308). If the motion information and the color image information correspond to the selected record (Yes at Step S308), the person determining unit 1405 increments the retained assistant characteristic number m by 1 (Step S309). The person determining unit 1405 then determines whether the retained assistant characteristic number m reaches 5 (Step S310). If the retained assistant characteristic number m reaches 5 (Yes at Step S310), the person determining unit 1405 determines that the person corresponding to the motion information acquired by the acquiring unit 1404 is the assistant (Step S311).

By contrast, if the retained assistant characteristic number m does not reach 5 (No at Step S310), the person determining unit 1405 determines whether an unprocessed record is present in the assistant motion characteristic storage unit and the assistant image characteristic storage unit (Step S312). If an unprocessed record is present (Yes at Step S312), the person determining unit 1405 repeats the processing from Step S307.

By contrast, if no unprocessed record is present (No at Step S312), the person determining unit 1405 determines that the person corresponding to the motion information acquired by the acquiring unit 1404 cannot be determined (Step S313).

As described above, the motion information processing apparatus 100a according to the fifth embodiment acquires motion information indicating a motion of a person. The motion information processing apparatus 100a detects an assistance state indicating a state created by the assistant for the object person serving as a target of rehabilitation based on the acquired motion information. The motion information processing apparatus 100a outputs assistance support information used to support the assistant based on the detected assistance state. As a result, the motion information processing apparatus 100a can increase the quality of assistance given by the assistant.

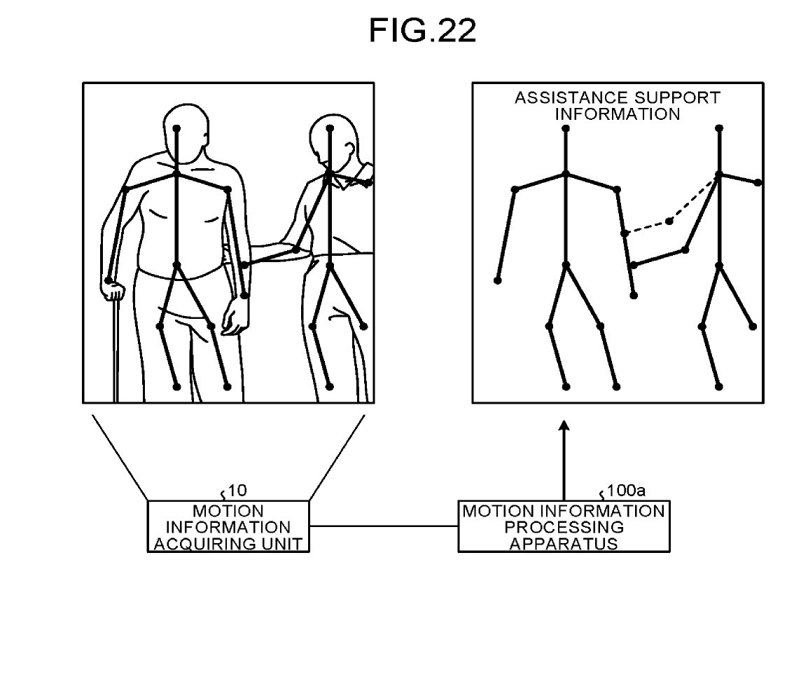

FIG. 22 is a view for explaining advantageous effects of the motion information processing apparatus 100a according to the fifth embodiment. As illustrated in FIG. 22, the motion information processing apparatus 100a acquires motion information indicating motions of the object person and the assistant in rehab. The motion information processing apparatus 100a detects an assistance state indicating a state created by the assistant for the object person from the acquired motion information. The motion information processing apparatus 100a outputs assistance support information 15a based on the detected assistance state. Specifically, the motion information processing apparatus 100a presents the assistance support information 15a in which the current assistance state is represented with the solid lines and recommended positions of joints are represented with the dashed lines to the assistant, for example. Thus, the motion information processing apparatus 100a can maintain the quality of assistance constant even if an unskilled assistant gives assistance, for example.

### Sixth Embodiment

While the fifth embodiment has described the case where the assistance given by the assistant to the object person is supported, the embodiment is not limited thereto. The motion information processing apparatus 100a may be used in a case where the assistant assists the object person using a device, for example. A sixth embodiment describes processing performed when the motion information processing apparatus 100a is used in the case where the assistant assists the object person using a device.

The sixth embodiment describes an example in which a assistant helps an object person who cannot stand up by himself/herself with a standing-up motion using an assistance belt. The assistance belt is worn by the assistant. The assistant causes the object person to hold the assistance belt worn by the assistant when the object person stands up, thereby helping the object person with the standing-up motion.

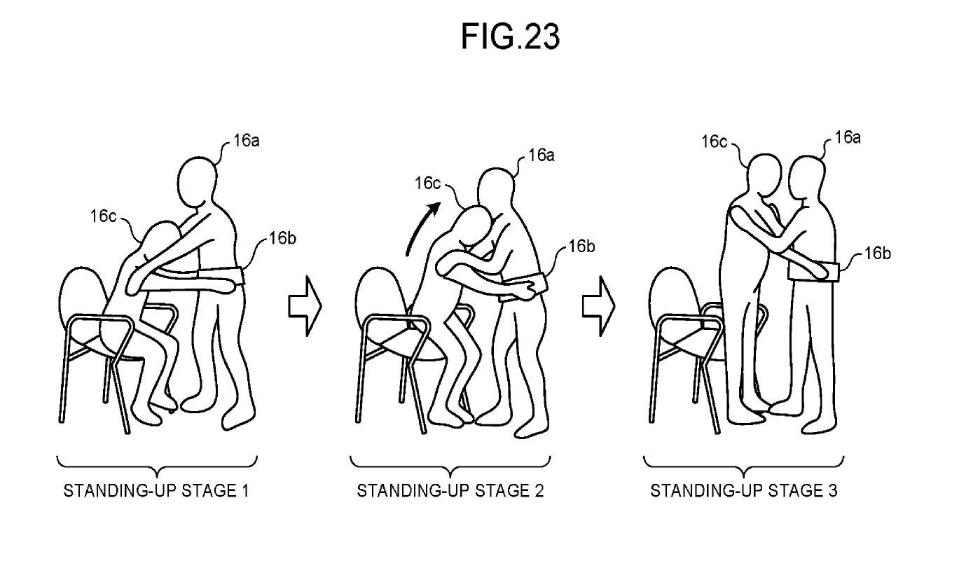

FIG. 23 is a view for explaining a case where an assistant helps an object person with a standing-up motion using an assistance belt. As illustrated in FIG. 23, assistance stages of a standing-up motion with an assistance belt include three stages of assistance stages 1, 2, and 3 performed in order, for example. At the standing-up stage 1, an assistant 16a wears an assistance belt 16b on the waist and stands in front of an object person 16c. At this time, the object person sits with the hands holding the assistance belt 16b worn by the assistant 16a. At the standing-up stage 2, the object person 16c starts the standing-up motion from the state of the standing-up stage 1. At the standing-up stage 3, the standing-up motion of the object person 16c is completed from the state of the standing-up stage 2, and the object person 16c stands. Thus, the assistance belt 16b is worn by the assistant 16a, thereby helping the object person 16c with the standing-up motion.

A motion information processing apparatus 100a according to the sixth embodiment supports the assistant 16a who assists the object person 16c with the standing-up motion using the assistance belt 16b by the processing described below. While the sixth embodiment describes the case of supporting the assistant 16a assisting the object person 16c with the standing-up motion using the assistance belt 16b, the embodiment is not limited thereto. The embodiment is also applicable to a case where the assistant assists the object person using another device.

The motion information processing apparatus 100a according to the sixth embodiment has the same configuration as that of the motion information processing apparatus 100a illustrated in FIG. 12 but is different therefrom in a part of information stored in a recommended assistance state storage unit 1307 and processing performed by a detecting unit 1407, an output determining unit 1408, and an output control unit 1409. The sixth embodiment mainly describes differences from the fifth embodiment. Points having functions equivalent to those in the configuration described in the fifth embodiment are denoted by the same reference numerals as those in FIG. 12, and the explanation thereof will be omitted.

The recommended assistance state storage unit 1307 stores therein an assistance state further including a positional relation of a device used by the assistant to assist the object person.

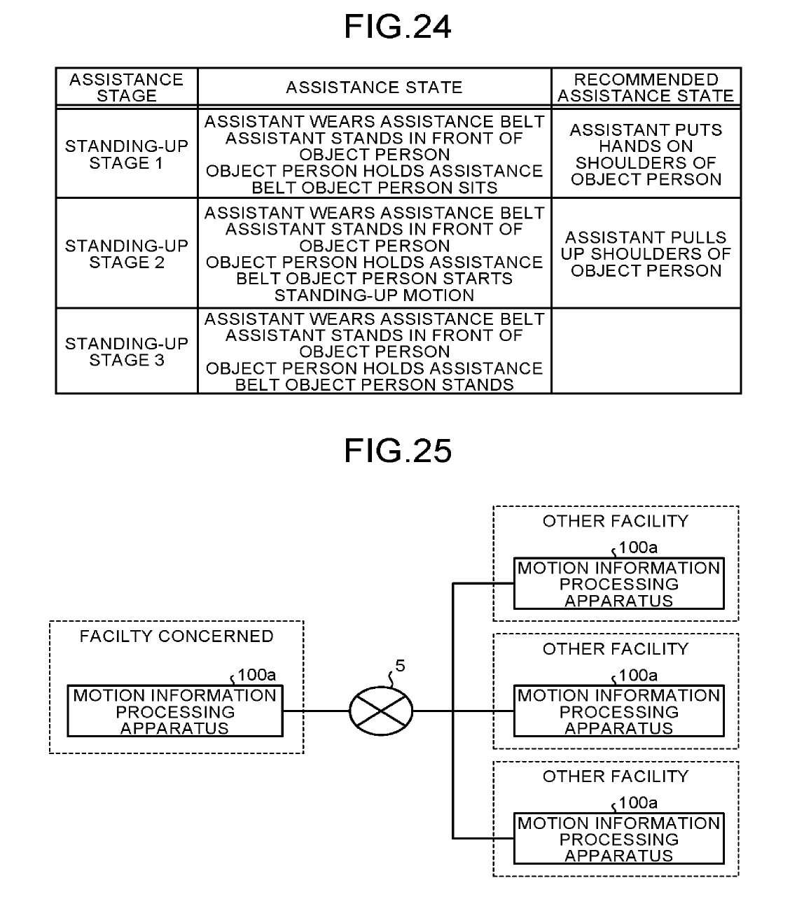

FIG. 24 is a diagram of an example of information stored in the recommended assistance state storage unit 1307. FIG. 24 illustrates an example in which the recommended assistance state storage unit 1307 stores therein recommended assistance states relating to the standing-up motion illustrated in FIG. 23. In the first record in FIG. 24, the assistance stage “standing-up stage 1”, assistance states of “the assistant wears the assistance belt”, “the assistant stands in front of the object person”, “the object person holds the assistance belt”, and “the object person sits”, and a recommended assistance state “the assistant puts the hands on the shoulders of the object person” are associated with one another. In other words, the recommended assistance state storage unit 1307 stores therein information that the assistance stage “standing-up stage 1” in the standing-up motion illustrated in FIG. 23 corresponds to the state where “the assistant wears the assistance belt”, “the assistant stands in front of the object person”, “the object person holds the assistance belt”, and “the object person sits”, and that a recommended motion made by the assistant for the object person is “the assistant puts the hands on the shoulders of the object person”. In the second record in FIG. 24, the assistance stage “standing-up stage 2”, assistance states of “the assistant wears the assistance belt”, “the assistant stands in front of the object person”, “the object person holds the assistance belt”, and “the object person starts the standing-up motion”, and a recommended assistance state “the assistant pulls up the shoulders of the object person” are associated with one another. In other words, the recommended assistance state storage unit 1307 stores therein information that the assistance stage “standing-up stage 2” in the standing-up motion illustrated in FIG. 23 corresponds to the state where “the assistant wears the assistance belt”, “the assistant stands in front of the object person”, “the object person holds the assistance belt”, and “the object person starts the standing-up motion”, and that a recommended motion made by the assistant for the object person is “the assistant pulls up the shoulders of the object person”. In the third record in FIG. 24, the assistance stage “standing-up stage 3”, assistance states of “the assistant wears the assistance belt”, “the assistant stands in front of the object person”, “the object person holds the assistance belt”, and “the object person stands” are associated with each other, and no information is stored in the recommended assistance state. In other words, the recommended assistance state storage unit 1307 stores therein information that the assistance stage “standing-up stage 3” in the standing-up motion illustrated in FIG. 23 corresponds to the state where “the assistant wears the assistance belt”, “the assistant stands in front of the object person”, “the object person holds the assistance belt”, and “the object person stands” and that no recommended motion made by the assistant for the object person is present.

The detecting unit 1407 extracts device characteristic information indicating characteristics of the device used by the assistant to assist the object person from color image information acquired by an acquiring unit 1404. The detecting unit 1407 then uses the extracted device characteristic information to detect an assistance state. The detecting unit 1407, for example, performs pattern matching of the assistance belt in the color image information with an image pattern of the assistance belt, thereby acquiring coordinate information on the assistance belt and information indicating the direction of the assistance belt. The detecting unit 1407 uses the acquired coordinate information on the assistance belt and information indicating the direction of the assistance belt, thereby detecting an assistance state. The detecting unit 1407, for example, uses the coordinate information and the directions of the object person, the assistant, and the device, thereby detecting one of or a combination of a positional relation, movement states, an assistance action, and explicit actions as an assistance state. If the detecting unit 1407 detects an assistance state using color image information and distance image information on the standing-up stage 2 in FIG. 23, for example, the detecting unit 1407 detects assistance states of “the assistant wears the assistance belt”, “the assistant stands in front of the object person”, “the object person holds the assistance belt”, “the object person starts the standing-up motion”, and “the assistant pulls up the shoulders of the object person”.

The output determining unit 1408 determines whether the assistance state detected by the detecting unit 1407 satisfies a recommended assistance state. The output determining unit 1408, for example, receives the assistance states of “the assistant wears the assistance belt”, “the assistant stands in front of the object person”, “the object person holds the assistance belt”, “the object person starts the standing-up motion”, and “the assistant pulls up the shoulders of the object person”. The output determining unit 1408 then refers to the recommended assistance state storage unit 1307. Because the received assistance states satisfies all the four states included in the assistance states of “the assistant wears the assistance belt”, “the assistant stands in front of the object person”, “the object person holds the assistance belt”, and “the object person starts the standing-up motion”, the output determining unit 1408 identifies the assistance stage corresponding to the received assistance states as the assistance stage “standing-up stage 2”. The output determining unit 1408 compares the received assistance states with the recommended assistance state of the “standing-up stage 2”, that is, “the assistant pulls up the shoulders of the object person”. Because the assistant 12b pulls up the shoulders of the object person 12a, the output determining unit 1408 determines that the received assistance states satisfy the recommended assistance state.

The output control unit 1409 outputs assistance support information based on the device characteristic information and the assistance state detected by the detecting unit 1407. The output control unit 1409, for example, outputs assistance support information used to support the assistant based on the assistance state detected by the detecting unit 1407.

As described above, the motion information processing apparatus 100a according to the sixth embodiment acquires color image information corresponding to motion information. The motion information processing apparatus 100a detects device characteristic information indicating characteristics of a device used by the assistant to assist the object person from the acquired color image information. The motion information processing apparatus 100a outputs assistance support information based on the detected device characteristic information and an assistance state. Thus, the motion information processing apparatus 100a can improve the quality of assistance given by the assistant by outputting appropriate assistance support information to the assistant in the case where the assistant assists the object person using a device.

The embodiment is not limited to the example described above. The embodiment may be applied to a case where the assistant wears an assistance belt, for example. Specifically, the motion information processing apparatus 100a defines a state where the assistance belt is correctly worn as a wearing stage and stores therein a positional relation between the assistant and the assistance belt worn by the assistant. The motion information processing apparatus 100a acquires the skeletal information on the assistant and the coordinate information on the assistance belt and the information indicating the direction of the assistance belt (including a relative distance, for example) acquired by pattern matching. The motion information processing apparatus 100a compares the acquired information with the positional relation of the wearing stage. If the acquired positional relation between the assistant and the assistance belt is different from the positional relation of the wearing stage, the motion information processing apparatus 100a outputs a warning sound and information indicating a correct wearing position to the assistant. If the position of the assistance belt worn by the assistant is higher than that in the positional relation of the wearing stage, for example, the motion information processing apparatus 100a notifies the assistant of information indicating how much the assistance belt has to be lowered together with a warning sound.

The motion information processing apparatus 100a is also applicable to “a safety state of a walking support device (a position of a walking support device)”, “the length and an angle of walking poles and how to move the walking poles”, “positions of a toilet and an excretion tool”, “a position of a bathing related tool (a shower chair, a bath board, and the like)”, “an assistance position and a work method on a bed (prevention of bedsore, decubitus, and the like)”, “positions of other daily necessaries”, and “a method for using a weight for muscle building training (for both objects and persons)”, for example. The operator, for example, classifies a series of motions relating to the usage described above into several stages and stores information defining the states of a person and a device at each stage in a storage unit 130 of the motion information processing apparatus 100a. Thus, the motion information processing apparatus 100a is applied to a case where assistance is given using the devices described above.

### Seventh Embodiment

A seventh embodiment describes a case where the motion information processing apparatus 100a in a facility concerned supports assistance given by the assistant to the object person using a recommended assistance state storage units 1307 used in other facilities in addition to the embodiments described above.

FIG. 25 is a diagram of an example of an entire configuration of the motion information processing apparatus 100a according to the seventh embodiment. As illustrated in FIG. 25, the motion information processing apparatus 100a provided in a facility concerned is connected to motion information processing apparatuses 100a of other facilities via a network 5. The motion information processing apparatuses 100a provided in the facility concerned and the other facilities each have a public storage unit that can be accessed from the facilities other than the facilities they belong to. The public storage unit stores therein the recommended assistance state storage unit 1307. The network 5 may be any desired type of wired or wireless communication network, such as the Internet, a local area network (LAN), and a virtual private network (VPN). While the motion information processing apparatus 100a in the facility concerned is connected to the motion information processing apparatuses 100a in the other facilities in the example of FIG. 25, the embodiment is not limited thereto. The motion information processing apparatus 100a in the facility concerned may be directly connected to the public storage units in the other facilities, for example. The motion information processing apparatus 100a may be directly connected to public storage units managed by academic organizations, third-party organizations, or service providers, for example.

FIG. 26 is a block diagram of an exemplary configuration of the motion information processing apparatus 100a according to the seventh embodiment. The motion information processing apparatus 100a illustrated in FIG. 26 is different from the motion information processing apparatus 100a illustrated in FIG. 12 in that a recommended assistance state acquiring unit 1410 is further provided. The seventh embodiment mainly describes differences from the fifth embodiment. Points having functions equivalent to those in the configuration described in the fifth embodiment are denoted by the same reference numerals as those in FIG. 12, and the explanation thereof will be omitted.

The recommended assistance state acquiring unit 1410 acquires a recommended assistance state indicating a state of assistance recommended when the assistance is given by the assistant. The recommended assistance state acquiring unit 1410, for example, receives a search request for searching the recommended assistance state storage units 1307 in the other facilities relating to gait training from the user. The search request specifies information on a facility, a state of a patient, or a type of rehab as a search key, for example. If the search request is received, the recommended assistance state acquiring unit 1410 acquires a list of recommended assistance states corresponding to the search request from the recommended assistance state storage units 1307 stored in the public storage units of the motion information processing apparatuses 100a in the other facilities. The recommended assistance state acquiring unit 1410 notifies the user of the acquired list of recommended assistance states. If the user selects one or a plurality of recommended assistance states from the list, the recommended assistance state acquiring unit 1410 acquires the selected recommended assistance states from the recommended assistance state storage units 1307. The recommended assistance state acquiring unit 1410 then stores the acquired recommended assistance states relating to gait training in the recommended assistance state storage unit 1307 in the facility concerned as other facility recommended assistance states in a manner separated from the recommended assistance state in the facility concerned and associated with the assistance stages.

An output determining unit 1408 compares the other facility recommended assistance state acquired by the recommended assistance state acquiring unit 1410 with an assistance state detected by a detecting unit 1407, thereby determining whether the assistance state satisfies the recommended assistance state. The output determining unit 1408, for example, receives an assistance state detected by the detecting unit 1407. The output determining unit 1408 refers to the recommended assistance state storage unit 1307, thereby identifying an assistance stage corresponding to the received assistance state. The output determining unit 1408 compares the received assistance state with the other facility recommended assistance state corresponding to the identified assistance stage, thereby determining whether the assistance state satisfies the other facility recommended assistance state. The output determining unit 1408 outputs the determination result to an output control unit 1409.

The output control unit 1409 outputs assistance support information based on the result of comparison made by the output determining unit 1408. If the output control unit 1409 receives a determination result indicating that the assistance state detected by the detecting unit 1407 does not satisfy the other facility recommended assistance state from the output determining unit 1408, for example, the output control unit 1409 causes an output unit 110 to display a display image displaying the other facility recommended assistance state and the recommended assistance state in the facility concerned in a manner arranged side by side.

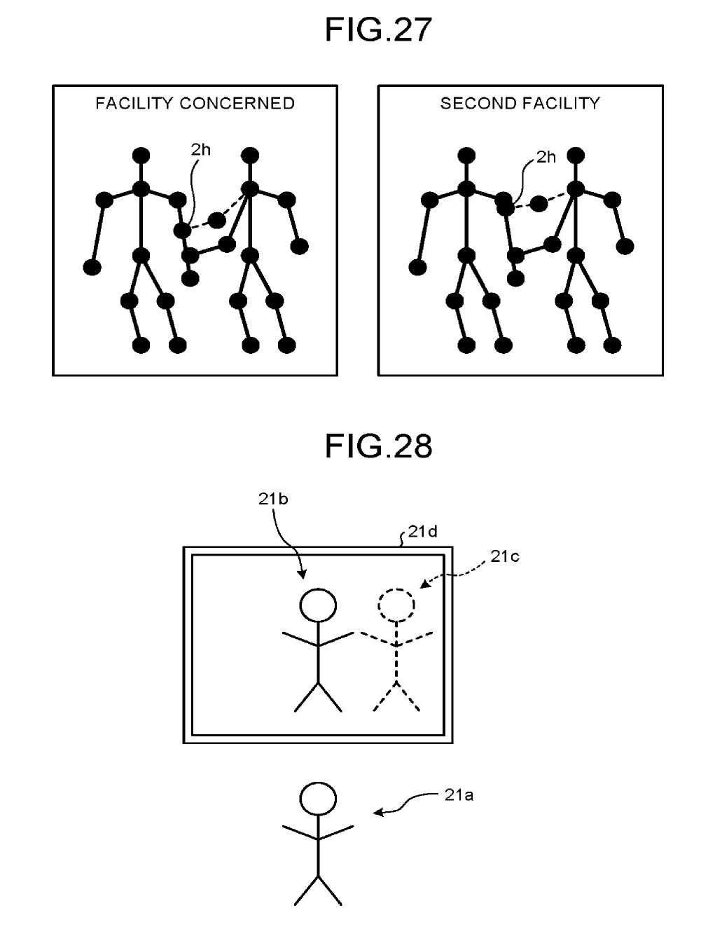

FIG. 27 is a view for explaining processing of the output control unit 1409 according to the seventh embodiment. In FIG. 27, the solid lines represent an assistance state, and the dotted lines represent a recommended assistance state. The left figure of FIG. 27 is an image illustrating a recommended assistance state and an assistance state in the facility concerned, whereas the right figure of FIG. 27 is an image illustrating a recommended assistance state and an assistance state in a second facility. As illustrated in FIG. 27, the output control unit 1409 outputs a display image displaying these images in a manner arranged side by side to a monitor. This can facilitate the assistant's finding that the position of the right hand (joint 2h) indicated in the recommended assistance state in the second facility is higher than the position of the right hand (joint 2h) indicated in the recommended assistance state in the facility concerned.

While the explanation has been made of the case where the image displaying the recommended assistance state and the assistance state in the facility concerned and the image displaying the recommended assistance state and the assistance state in the second facility are displayed side by side in FIG. 27, the embodiment is not limited thereto. The output control unit 1409 may display these two images in a superimposed manner, for example. The output control unit 1409 may calculate a gap between the position of the right hand (joint 2h) indicated in the recommended assistance state in the facility concerned and the position of the right hand (joint 2h) indicated in the recommended assistance state in the second facility and then notify the assistant of the calculated gap as a numerical value, for example. The output control unit 1409 may statistically calculate, for example, the average value of the positions of the right hand indicated in the recommended assistance states of a plurality of other facilities and then notify the assistant of the calculated value.

As described above, the motion information processing apparatus 100a according to the seventh embodiment acquires a recommended assistance state indicating a state of assistance recommended when the assistance is given by the assistant. The motion information processing apparatus 100a compares the acquired recommended assistance state and an assistance state, thereby outputting assistance support information based on the comparison result. Thus, the motion information processing apparatus 100a can support the assistance given by the assistant to the object person with the recommended assistance state storage units 1307 used in other facilities. This enables the motion information processing apparatus 100a to collect the best practice of the recommended assistance states used in the other facilities and use the best practice to support the assistant, for example.

The motion information processing apparatus 100a may include a functional unit that manages access restriction, for example. The motion information processing apparatus 100a may permit access from a motion information processing apparatus 100a of a specific facility or restrict access from a motion information processing apparatus 100a of another specific facility, for example.

The motion information processing apparatus 100a may include a functional unit that manages access history, for example. The motion information processing apparatus 100a may store therein the access history in a manner associated with evaluation from the other facilities. The motion information processing apparatus 100a may store the recommended assistance state storage unit 1307 in the public storage unit together with information indicating approval of a doctor, evidence, and recommendation from a medical professional, for example. The motion information processing apparatus 100a may charge the other facilities for each acquisition of the recommended assistance state storage unit 1307 carried out by the motion information processing apparatuses 100a in the other facilities, for example.

The motion information processing apparatus 100a may update the recommended assistance state used to support the assistant in the facility concerned with the acquired other facility recommended assistance state, thereby importing the other facility recommended assistance state as information in the facility concerned, for example.

The motion information processing apparatus 100a may include a system that gives feedback to a source facility about the acquired recommended assistance state, for example. In the acquisition of the recommended assistance state, for example, the motion information processing apparatus 100a stores therein information indicating a source facility of the recommended assistance state together with the recommended assistance state. The motion information processing apparatus 100a receives input of information on feedback (remarks and evaluation) about the acquired recommended assistance state from the object person, the assistant, or the operator, for example. The motion information processing apparatus 100a can transmit the received information on feedback to the motion information processing apparatus 100a of the source facility of the recommended assistance state to which the feedback has been given.

### Eighth Embodiment

An eighth embodiment describes a case where the motion information processing apparatuses 100a explained in the embodiments described above are used for education of a assistant.

A motion information processing apparatus 100a according to the eighth embodiment has the same configuration as that of the motion information processing apparatus 100a illustrated in FIG. 12 but is different therefrom in a part of processing performed by a detecting unit 1407, an output determining unit 1408, and an output control unit 1409. The eighth embodiment mainly describes differences from the fifth embodiment. Points having functions equivalent to those in the configuration described in the fifth embodiment are denoted by the same reference numerals as those in FIG. 12, and the explanation thereof will be omitted. The motion information processing apparatus 100a according to the eighth embodiment does not necessarily include the mode determining unit 1406.

The detecting unit 1407 detects a state of the object person serving as a target of rehabilitation or a state of the assistant who assists the object person based on motion information acquired by an acquiring unit 1404. The detecting unit 1407, for example, uses one of or a combination of a positional relation, movement states, an assistance action, and explicit actions, thereby detecting the state of the object person or the state of the assistant. The detecting unit 1407 outputs the state of the object person or the state of the assistant detected to the output determining unit 1408. As described above, the processing of the detecting unit 1407 is performed on the motion information in which at least one object person and one assistant are identified by a person determining unit 1405. In the following description, an assumption is made that the object person and the assistant are identified.

If the detecting unit 1407 detects the state of the object person, for example, the output determining unit 1408 acquires information indicating the state of the assistant assisting the object person from a recommended assistance state storage unit 1307. If the detecting unit 1407 detects the state of the assistant, for example, the output determining unit 1408 acquires information indicating the state of the object person being assisted by the assistant from the recommended assistance state storage unit 1307.

The processing of the output determining unit 1408 will be described with reference to FIG. 24. If a state of the object person that “the object person holds the assistance belt, and the object person sits” is detected by the detecting unit 1407, for example, the output determining unit 1408 refers to the recommended assistance state storage unit 1307 in FIG. 24, thereby identifying the assistance stage corresponding to the detected assistance state as the assistance stage “standing-up stage 1”. The output determining unit 1408 refers to the assistance state and the recommended assistance state of the assistance stage “standing-up stage 1”, thereby acquiring information indicating the state of the assistant assisting the object person. Specifically, the state of the assistant satisfies “the assistant wears the assistance belt”, “the assistant stands in front of the object person”, and “the assistant puts the hands on the shoulders of the object person” out of the assistance states of “the assistant wears the assistance belt”, “the assistant stands in front of the object person”, “the object person holds the assistance belt”, and “the object person sits”, and the recommended assistance state “the assistant puts the hands on the shoulders of the object person”. The output determining unit 1408 acquires these pieces of information as information indicating the state of the assistant. The output determining unit 1408 outputs the acquired information indicating the state of the assistant to the output control unit 1409. To acquire information indicating the state of the object person being assisted by the assistant from the state of the assistant, the output determining unit 1408 performs the processing in the same manner as described above except that the assistant is replaced with the object person. Thus, the explanation thereof will be omitted.

If the detecting unit 1407 detects the state of the object person, the output control unit 1409 outputs the information indicating the state of the assistant assisting the object person. If the detecting unit 1407 detects the state of the assistant, the output control unit 1409 outputs the information indicating the state of the object person being assisted by the assistant. The output control unit 1409, for example, outputs the information indicating the state of the object person or the information indicating the state of the assistant received from the output determining unit 1408 to an output unit 110.

FIG. 28 is a view for explaining the processing of the output control unit 1409 according to the eighth embodiment. In FIG. 28, a person 21a performs a role of an object person, thereby checking a motion of an assistant on a screen 21. The person 21a is a person who is trained as an assistant, for example. If the person 21a performs the role of the object person and makes motions similar to the states of the object person of “the object person holds the assistance belt” and “the object person sits”, the screen 21 displays a person image 21b that makes motions similar thereto. A screen 21d displays a virtual person image 21c generated based on an assistance state and a recommended assistance state corresponding to the assistance stage of the person image 21b. The virtual person image 21c performs the role of the assistant and represents the state of the assistant corresponding to the state of the object person expressed by the person 21a.

As described above, the motion information processing apparatus 100a according to the eighth embodiment acquires motion information indicating a motion of a person. The motion information processing apparatus 100a detects a state of the object person serving as a target of rehabilitation or a state of the assistant who assists the object person based on the acquired motion information. If the state of the object person is detected, the motion information processing apparatus 100a outputs information indicating a state of the assistant assisting the object person. If the state of the assistant is detected, the motion information processing apparatus 100a outputs information indicating a state of the object person being assisted by the assistant. When a person performs a role of the object person or the assistant, the motion information processing apparatus 100a can output information indicating a state of the assistant or the object person at each assistance stage, thereby enabling the person to perform simulation assistance.

While the explanation has been made of the case where the recommended assistance state is used as a good example of the assistance motion for education of a assistant in the eighth embodiment, the embodiment is not limited thereto. The recommended assistance state storage unit 1307 may register therein bad examples of the assistance motion, and the bad examples may be presented for education of a assistant, for example. Specifically, the recommended assistance state storage unit 1307 registers therein assistance states of typical bad examples corresponding to years of experience as an assistant, such as a person inexperienced in assistance, a person having 0 to 1 year of experience in assistance, and a person having 1 to 3 years of experience in assistance, and attributes of functional levels, for example. With this configuration, the motion information processing apparatus 100a can use the bad examples of the assistance motion for education of an assistant.

The motion information processing apparatus 100a may include a system that evaluates an assistance motion for education of an assistant, for example. The motion information processing apparatus 100a, for example, compares an assistance motion made by a trainee who takes training as an assistant with the recommended assistance state using the function described in the fifth embodiment. The motion information processing apparatus 100a derives a gap between the assistance motion and the recommended assistance state, thereby evaluating the trainee. The motion information processing apparatus 100a may give a score represented by 0 to 100 points based on the derived gap. The motion information processing apparatus 100a may accumulate scores of a plurality of trainees and rank the trainees in descending order of their scores accumulated.

### Ninth Embodiment

A ninth embodiment describes a case where the motion information processing apparatus 100a supports the assistance given by the assistant to the object person using information received from other sensors in addition to the processing of the motion information processing apparatus 100a described in the embodiments described above.

A motion information processing apparatus 100a according to the ninth embodiment has the same configuration as that of the motion information processing apparatus 100a illustrated in FIG. 12 but is different therefrom in a part of processing performed by an acquiring unit 1404 and an output control unit 1409. The ninth embodiment mainly describes differences from the fifth embodiment. Points having functions equivalent to those in the configuration described in the fifth embodiment are denoted by the same reference numerals as those in FIG. 12, and the explanation thereof will be omitted.

The acquiring unit 1404 acquires biological information on the object person. In the case where a sphygmomanometer and a pulse meter are used for an input unit 120, for example, the acquiring unit 1404 uses these devices to acquire the blood pressure and pulse of the object person. The acquiring unit 1404 outputs the acquired biological information on the object person to an output determining unit 1408. The biological information acquired by the acquiring unit 1404 can be associated with frames included in motion information using acquisition time at which the biological information was acquired.

The output control unit 1409 outputs assistance support information based on the biological information acquired by the acquiring unit 1404 and an assistance state. If the blood pressure of the object person drops at the assistance stage 1, for example, the output control unit 1409 outputs information indicating a recommended assistance state corresponding to the assistance stage 2 to the assistant. This enables the assistant to know a recommended assistance motion in advance and quickly respond to the object person who is getting sick.

As described above, the motion information processing apparatus 100a according to the ninth embodiment acquires biological information on the object person. The motion information processing apparatus 100a outputs assistance support information based on the acquired biological information and an assistance state. Thus, the motion information processing apparatus 100a can support the assistance given by the assistant to the object person based on a change in the biological information on the object person.

The ninth embodiment is not limited to the example described above. Biological information on the object person in a normal state may be compared with the current biological information, for example. Specifically, in this case, the motion information processing apparatus 100a stores therein biological information on the object person in a normal state (e.g., normal blood pressure and normal pulse). The motion information processing apparatus 100a compares acquired current blood pressure and the stored normal blood pressure. If the current blood pressure is lower than the normal blood pressure, the motion information processing apparatus 100a notifies the assistant of the fact, thereby allowing the assistant to give more careful assistance than usual.

### Other Embodiments

While the first to the ninth embodiments have been described, the embodiments may be implemented in various different forms other than the foregoing first to the ninth embodiments.

In the first to the fourth embodiments, the explanation has been made of the case where the motion information processing apparatus 100 acquires rule information corresponding to the object person based on a disease part of the object person, determines whether a motion corresponding to motion information on the object person follows the rules, and notifies the object person of the determination result. The embodiments are not limited thereto, and each processing may be performed by a service providing apparatus on a network, for example.

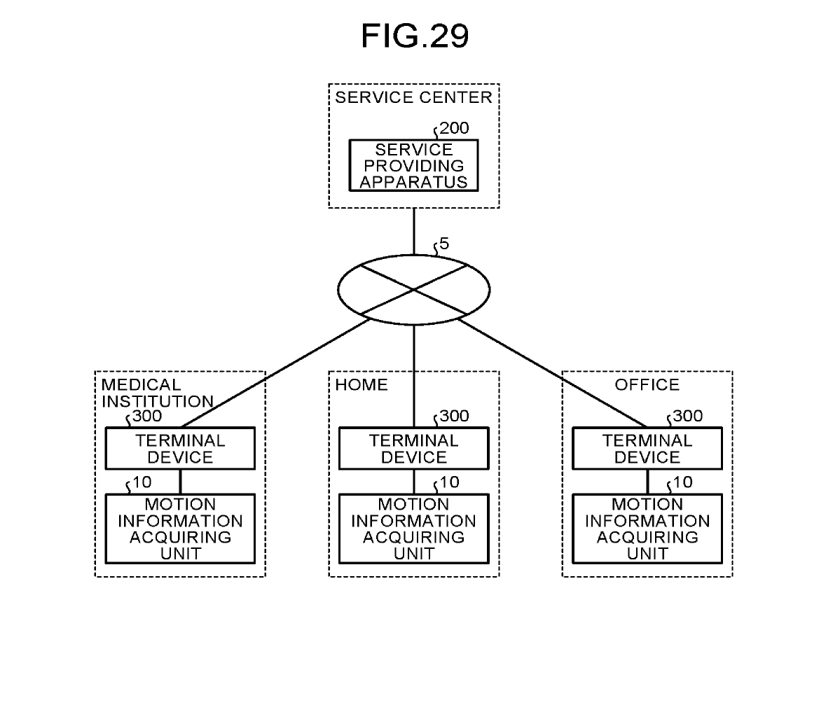

FIG. 29 is a view for explaining an example of a case where the embodiments are applied to a service providing apparatus. As illustrated in FIG. 29, a service providing apparatus 200 is placed in a service center and is connected to terminal devices 300 placed in a medical institution, home, and an office via a network 5, for example. The terminal devices 300 placed in the medical institution, home, and the office are each connected to a motion information acquiring unit 10. The terminal devices 300 each have a client function to use services provided from the service providing apparatus 200.

The service providing apparatus 200 provides processing similar to that performed by the motion information processing apparatus 100 illustrated in FIG. 4 to the terminal devices 300 as a service. In other words, the service providing apparatus 200 includes functional units similar to the acquiring unit 1401, the determining unit 1402, and the output control unit 1403. The functional unit similar to the acquiring unit 1401 acquires motion information relating to the skeletal structure of the object person serving as a target of rehabilitation. The functional unit similar to the determining unit 1402 determines whether a motion of the object person indicated by the motion information acquired by the functional unit similar to the acquiring unit 1401 follows regulations included in rule information based on the rule information on the object person in rehabilitation. The functional unit similar to the output control unit 1403 performs control to output the result of determination made by the functional unit similar to the determining unit 1402. The network 5 may be any desired type of wired or wireless communication network, such as the Internet and a wide area network (WAN).

The configuration of the motion information processing apparatus 100 according to the first to the fourth embodiments is given by way of example, and the units may be integrated and separated as appropriate. The object person information storage unit 1302 and the rule information storage unit 1303 may be integrated, for example. The determining unit 1402 may be separated into an extracting unit that extracts rule information corresponding to the object person and a determining unit that determines a motion, for example.

The functions of the acquiring unit 1401, the determining unit 1402, and the output control unit 1403 described in the first to the fourth embodiments may be carried out by software. The functions of the acquiring unit 1401, the determining unit 1402, and the output control unit 1403 are carried out by a computer executing a medical information processing program specifying the procedure of the processing described as processing performed by the acquiring unit 1401, the determining unit 1402, and the output control unit 1403 in the embodiments, for example. The medical information processing program is stored in a hard disk or a semiconductor memory device, for example, and is read and executed by a processor, such as a CPU and a micro processing unit (MPU). The medical information processing program may be recorded and distributed in a computer-readable recording medium, such as a CD-ROM, an MO, and a DVD.

In the fifth to the ninth embodiments, the explanation has been made of the case where the motion information processing apparatus 100a analyzes motion information acquired by the motion information acquiring unit 10, thereby supporting the object person and the assistant. The embodiments are not limited thereto, and each processing may be performed by a service providing apparatus on a network, for example.

The service providing apparatus 200 illustrated in FIG. 29 has functions similar to those of the motion information processing apparatus 100a illustrated in FIG. 12 and provides the functions to the terminal devices 300 as a service. In other words, the service providing apparatus 200 includes functional units similar to the acquiring unit 1404, the detecting unit 1407, and the output control unit 1409. The functional unit similar to the acquiring unit 1404 acquires motion information indicating a motion of a person. The functional unit similar to the detecting unit 1407 detects an assistance state indicating a state of the assistant for the object person serving as a target of rehabilitation based on the motion information acquired by the functional unit similar to the acquiring unit 1404. The functional unit similar to the output control unit 1409 outputs assistance support information used to support the assistant based on the assistance state detected by the functional unit similar to the detecting unit 1407. This can increase the quality of assistance given by the assistant.

The configuration of the motion information processing apparatus 100a according to the fifth to the ninth embodiments is given by way of example, and the units may be integrated and separated as appropriate. The object person motion characteristic storage unit 1305A and the assistant motion characteristic storage unit 1305B may be integrated, for example.

The rule information and the recommended assistance states in rehabilitation described in the first to the ninth embodiments may be defined by various organizations besides the Japanese Orthopaedic Association. Various types of regulations and rules may be used, including regulations and rules defined by the International Society of Orthopaedic Surgery and Traumatology (SICOT), the American Academy of Orthopaedic Surgeons (AAOS), the European Orthopaedic Research Society (EORS), the International Society of Physical and Rehabilitation Medicine (ISPRM), and the American Academy of Physical Medicine and Rehabilitation (AAPM&R), for example.

The motion information processing apparatus and the method according to at least one of the embodiments can increase the quality of rehabilitation.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A motion information processing apparatus comprising:
processing circuitry configured to
acquire motion information indicating a motion of a person, and
output support information used to support a motion relating to rehabilitation for the person whose motion information is acquired.

2. The motion information processing apparatus according to claim 1, wherein
the processing circuitry is further configured to
determine, based on rule information on an object person serving as a target of the rehabilitation, whether a motion of the object person indicated by the motion information acquired by the acquiring unit follows a regulation included in the rule information in the rehabilitation,
acquire motion information relating to a skeletal structure of the object person serving as a target of the rehabilitation, and
output a result of determination made.

3. The motion information processing apparatus according to claim 2, wherein the processing circuitry is configured to determine, based on rule information determined by contents of the rehabilitation performed by the object person and information on an affected area of the object person, whether the motion of the object person indicated by the motion information follows the regulation included in the rule information.

4. The motion information processing apparatus according to claim 3, wherein
the processing circuitry is configured to
acquire motion information subsequent to execution of a motion corresponding to the contents of the rehabilitation, and
determine whether a motion indicated by the motion information subsequent to execution of the motion acquired follows the regulation included in the rule information.

5. The motion information processing apparatus according to claim 3, wherein
the processing circuitry is configured to
acquire motion information prior to execution of a motion corresponding to the contents of the rehabilitation, and
determine whether a motion indicated by the motion information prior to execution of the motion acquired follows the regulation included in the rule information.

6. The motion information processing apparatus according to claim 2, wherein
the processing circuitry is configured to
determine a motion following the regulation included in the rule information in contents of rehabilitation subsequently performed by the object person based on the motion indicated by the motion information acquired, and
output information on the motion determined to the object person.

7. The motion information processing apparatus according to claim 5, wherein
the processing circuitry is configured to
determine whether a motion being made by the object person is a motion following contents of rehabilitation being currently performed based on the motion information acquired, and
output, when the motion being made by the object person is determined to be not a motion following the contents of the rehabilitation being currently performed, information on a motion to return to the rehabilitation to the object person.

8. The motion information processing apparatus according to claim 2, wherein
the processing circuitry is configured to
acquire motion information on an object person who performs gait training,
determine, based on rule information determined by the gait training and information on a disease part of the object person, whether a motion indicated by the motion information on the object person who performs the gait training acquired follows the regulation included in the rule information, and
output a result of determination made to the object person who performs the gait training.

9. The motion information processing apparatus according to claim 8, wherein
the processing circuitry is configured to
acquire motion information on an object person who performs stair-climbing training of the gait training, and
determine, based on rule information determined by the stair-climbing training and information on a disease part of the object person, whether a motion indicated by the motion information on the object person who performs the stair-climbing training acquired follows the regulation included in the rule information.

10. The motion information processing apparatus according to claim 3, wherein the processing circuitry is configured to acquire the information on the disease part from at least one of a medical information system and a personal health record.

11. The motion information processing apparatus according to claim 1, wherein
the processing circuitry is further configured to
detect an assistance state created by an assistant for an object person serving as a target of the rehabilitation based on the motion information acquired, and
output assistance support information used to support the assistant based on the assistance state detected.

12. The motion information processing apparatus according to claim 11, wherein the assistance state includes at least one of a positional relation between the object person and the assistant, movement states of the object person and the assistant, and an instruction action performed by the assistant to the object person.

13. The motion information processing apparatus according to claim 11, wherein
the processing circuitry is configured to
acquire image information corresponding to the motion information, and
extract device characteristic information indicating characteristics of a device used by the assistant to assist the object person from the image information acquired and further uses the device characteristic information extracted to detect the assistance state.

14. The motion information processing apparatus according to claim 11, further comprising:
a recommended assistance state storage configured to store therein a recommended assistance state indicating a state of assistance recommended when the assistance is given by the assistant, wherein the processing circuitry is configured to compare the assistance state with the recommended assistance state and output the assistance support information based on a comparison result.

15. The motion information processing apparatus according to claim 14, wherein the processing circuitry is configured to calculate a difference between the assistance state and the recommended assistance state from the comparison result between the assistance state and the recommended assistance state, and output the calculated difference as the assistance support information.

16. The motion information processing apparatus according to claim 14, wherein
the processing circuitry is further configured to acquire the recommended assistance state from an external apparatus, compare the recommended assistance state acquired by the recommended assistance state acquiring unit with the assistance state and output the assistance support information based on a comparison result.

17. The motion information processing apparatus according to claim 11, wherein
the processing circuitry is configured to
acquire biological information on the object person, and
output the assistance support information based on the biological information acquired and the assistance state.

18. The motion information processing apparatus according to claim 11, wherein
the processing circuitry is configured to
detect a state of the object person serving as a target of the rehabilitation or a state of the assistant who assists the object person based on the motion information acquired, and
output, when the state of the object person is detected, information indicating a state of the assistant assisting the object person and output, when the state of the assistant is detected, information indicating a state of the object person being assisted by the assistant.

19. A method comprising:
acquiring motion information indicating a motion of a person; and
outputting support information used to support a motion relating to rehabilitation for the person whose motion information is acquired.
